(19) United States
(12) Patent Application Publication
(10) Pub. No.: US 2005/0043606 A1
(43) Pub. Date: Feb. 24, 2005

(54) Multiparametric apparatus for monitoring multiple tissue vitality parameters

(76) Inventors: Eliahu Pewzner, Modiin Ilit (IL); Avraham Mayevsky, Ramat Gan (IL); Alexander Vasilievitch Jaronkin, Rishon L'Zion (IL); Igor Derzy, Petach Tikvah (IL)

Correspondence Address: Kevin D McCarthy;Roach Brown McCarthy & Gruber, 1620 Liberty Building, Buffalo, NY, 14202, US

(21) Appl. No.: 10/490,674

(22) PCT Filed: Sep. 25, 2001
(86) PCT No.: PCT/IL01/00900

(51) Int. Cl. A61B 5/05

(52) U.S. Cl. 600/407

(57) ABSTRACT

Apparatus for monitoring a plurality of tissue viability parameters of a substantially identical tissue element, in which a single illumination laser source provides illumination radiation at a wavelength such as to enable monitoring of blood flow rate and NADH or flavoprotein concentration, together with blood volume and also blood oxygenation state. In preferred embodiments, an external cavity laser diode system is used to ensure that the laser operates in single mode or at else in two or three non-competing modes, each mode comprising a relatively narrow bandwidth. A laser stabilisation control system is provided to ensure long term operation of the laser source at the desired conditions.

### FIELD OF THE INVENTION

The present invention relates to apparatuses and methods for enabling simultaneous or individual monitoring of a plurality of tissue vitality parameters, particularly in-vivo, with respect to an identical tissue element, such parameters including blood flow rate, Mitochondrial Redox State via NADH or flavoprotein concentration, blood volume and blood oxygenation state. In particular, the present invention relates to such apparatuses and methods based on a single illuminating laser radiation.

### BACKGROUND OF THE INVENTION

Mammalian tissues are dependent upon the continuous supply of oxygen and glucose needed for the energy production. This energy is used for various types of work, including the maintaining of ionic balance and biosynthesis of various cellular components. The ratio or balance, between oxygen supply and demand reflects the cells' functional capacity to perform their work. In this way, the energy balance reflects the metabolic state of the tissue. In order to assess the tissue energy balance, it is necessary to monitor the events continuously using a multiparametric system in real-time.

The integrated system of energy supply and demand can be understood by considering the various components thereof.

O₂ supply: The blood carries the oxygen and other essential substances to the cells. Therefore, monitoring of blood flow rate, blood volume and blood oxygenation will reflect the supply of O₂ to the tissue for the purpose of energy formation therein.

Energy production and demand: In an inner compartment of the cells, called the mitochondria, the glucose and O₂ are transformed into ATP, a form of energy which can be used by the cells for various types of activities. The ATP production rate is, in normal states, regulated by rate of consumption of ATP, and is increased when cellular activity rises. In most pathological states, the limiting factor for this process is O₂ availability.

The process of energy (ATP) production and consumption can be determined through monitoring of Nicotineamide adenine dinucleotide (NADH) redox state. The NADH and NAD molecules can be correlated with the process of ATP production. The concentration of the reduced form of the molecule (NADH) rises when the rate of ATP production is low, and is unable to meet the demand in the tissue or cells.

A complementary indicator of energy production, other than NADH, is the concentration of flavoproteins (Fp). Flavoprotein molecules are also linked to the production of ATP in the mitochondria. Fp concentration drops when the rate of ATP production is reduced, and is unable to meet the demand in the tissue or cells.

There is a direct correlation between energy metabolism of the cellular compartment and the blood flow in the microcirculation of the same tissue. In a normal tissue, any change in the O₂ demand will be compensated by a corresponding change in the blood flow to the tissue. By this mechanism, the O₂ supply remains constant if there is no change in the O₂ consumption. Any change in the abundance of O₂ in the tissue, in other words a change in energy state, will be reflected by the NADH and Fp level.

It is important to monitor both supply and demand in order to be able to detect pathological situations in which the balance is disrupted, and one component of the system reacts abnormally with respect to the other.

The parameters used in the art for the assessment of tissue vitality include: A—Blood Flow Rate; B—Mitochondrial Redox State via the NADH level; C—Blood Volume; D—Blood Oxygenation State; E—Mitrochondrial Redox State via flavoprotein level.

A—Blood Flow Rate

The blood flow rate relates to the mean volume flow rate of the blood and is essentially equivalent to the mean velocity multiplied by the number of moving red blood cells in the tissue. This parameter may be monitored by a technique known as Laser Doppler Flowmetry, which is based on the fact that light reflected off moving red blood cells (RBC) undergoes a small shift in wavelength (Doppler shift) in proportion to the cell's velocity. Light reflected off of stationary RBC or bulk stationary tissue, on the other hand, does not undergo a Doppler shift.

By illuminating with coherent light, such as a laser, and converting the intensities of incident and reflected light to electrical signals, it is possible to estimate the blood flow from the magnitude and frequency distribution of those signals (U.S. Pat. No. 4,109,647; Stern, M. D. Nature 254, 56-58, 1975).

B—Mitochondrial Redox State or the NADH Level

The level of Nicotineamide adenine dinucleotide (NADH), the reduced form of NAD, is dependent both on the availability of oxygen and on the extent of tissue activity. Referring to FIG. 1, whilst NADH absorbs UV light at wavelengths of about 300 nm to about 400 nm and fluoresces at wavelengths of about 400 nm to about 550 nm, the NAD does not fluoresce. The NADH level can thus be measured using Mitochondrial NADH Fluorometry. The conceptual foundations for Mitochondrial NADH Fluorometry were established in the early 50's and were published by Chance and Williams (Chance B., & Williams G. R., Journal of Biological Chemistry, 217, 383-392, 1955). They defined various metabolic states of activity and rest for in-vitro mitochondria.

An increase in the level of NADH with respect to NAD and the resulting increase in fluorescence intensity indicate that insufficient Oxygen is being supplied to the tissue. Similarly, a decrease in the level of NADH with respect to NAD and the resulting decrease in fluorescence intensity indicate an increase in tissue activity.

C—Blood Volume

The blood volume parameter refers to the concentration of the blood in the tissue. When tissue is irradiated, the intensity R of reflection of the excitation wavelength light from the tissue is informative of the blood volume. The intensity R of the reflected signal, also referred to as the total backscatter, increases dramatically as blood is eliminated from the tissue as a result of the decrease in haemoglobin concentration. Similarly, if the tissue becomes more perfused with blood, R decreases due to the increase in the haemoglobin concentration.

D—Blood Oxygenation State

The blood oxygenation state parameter refers to the relative concentration of oxyhaemoglobin to deoxy-haemoglobin in the tissue. It may be assessed by the performance of photometry measurements. The absorption spectrum of oxyhaemoglobin HbO₂ is considerably different from the absorption spectrum of deoxy-haemoglobin Hb (Kramer R. S. and Pearlstein R. D., Science, 205, 693-696, 1979). The measurement of the absorption at one or more wavelengths can thus be used to assess this important parameter. Blood oximeters are based on measurement of the haemoglobin absorption changes as blood deoxygenates (Pologe J. A., Int. Anesthesiol. Clin., 25(3), 137-53, 1987). Such oximeters generally use at least two light wavelengths to probe the absorption. One known method uses one wavelength at an isosbestic point and another wavelength at a point that exhibits absorption changes due to variation in oxygenation level. Another technique uses wavelengths at both sides of an isosbestic point in order to increase measurement sensitivity. The wavelengths used in commercial pulse oximeters are typically around 660 nm in the red region of the spectrum, and between 800 nm to 1000 nm in near-infrared region (Pologe, 1987).

Isosbestic point as referred to herein is a wavelength at which the intensity of absorption of oxyhaemoglobin HbO₂ is the same as the intensity of absorption of deoxy-haemoglobin Hb; such isosbestic points are indicated as IPA and IPB in FIG. 3. Similarly, there is an isosbestic range marked IR in FIG. 3 where these two functions are substantially coincident. FIG. 3 is based on Anderson, R. R., Parrish, J. A. (1981) Microvasculature can be selectively damaged using dye lasers: a basic theory and experimental evidence in human skin. Lasers Surg.Med. 1, 263-276.

For monitoring the oxygenation levels of internal organs, fiber-optic blood oximeters have been developed. These fiber-optic devices irradiate the tissue with two wavelengths, and collect the reflected light. By analysis of the reflection intensities at several wavelengths the blood oxygenation is deduced. The wavelengths used in one such system were 585 nm (isosbestic point) and 577 nm (Rampil I. J., Litt L., & Mayevsky A., Journal of Clinical Monitoring, 8, 216-225, 1992). Another blood oximeter measures and analyzes the whole spectrum band 500-620 nm (Kessler M. & Frank K., Quantitative spectroscopy in tissue pp. 61-74. Verlagsgruppe GmbH, Frankfurt au Main, 1992). These devices are relatively complicated and susceptible to interference from ambient light, as well as various electronic and optic drifts. Two light sources are required, and the light sources and the detection system also incorporate optical filters that are interchangeable by mechanical means.

E—Flavoprotein Concentration

In order to determine the metabolic state of various tissues in-vivo it is also possible to monitor the fluorescence of another cellular fluorochrome, namely Flavoproteins (Fp). Referring to FIG. 12, Fp absorbs light at wavelengths of about 400 nm to about 470 nm and fluoresces at wavelengths of about 490 nm to about 580 nm. The Fp level can thus be measured using Fp Fluorometry. The conceptual foundations for Fp Fluorometry were established in the late 1960's and were published in several papers as will be referenced hereinafter. Simultaneous monitoring of NADH and Fp from the same layer or volume of tissue provides better interpretation of the changes in energy production and demand. Chance et al. (B. Chance, N. Graham, and D. Mayer. A time sharing fluorometer for the readout of intracellular oxidation-reduction states of NADH and Flavoprotein. The Review of Scientific Instruments 42 (7):951-957, 1971) used a time-sharing fluorometer to record intracellular redox state of NADH and Fp. They showed a very clear correlation between the two chromophores to changes in O₂ supply to the perfused liver. Using a time sharing fluorometer reflectometer simultaneous monitoring of NADH and Fp was performed from the surface of the rat's brain (A. Mayevsky. Brain energy metabolism of the conscious rat exposed to various physiological and pathological situations. Brain Res. 113:327-338, 1976). The kinetics of the responses to anoxia or decapitation were identical for the NADH and Fp indicating that the NADH signal comes from the same cellular compartment as the Fp—the mitochondrion.

The five tissue viability parameters described above represent various important biochemical and physiological activities of body tissues. Monitoring these parameters can provide much information regarding the tissues' vitality. For the monitoring of different parameters to have maximum utility however, the information regarding all parameters is required to originate from substantially the same layer of tissue, and preferably the same volume of tissue, otherwise misleading results can be obtained. In general, the more parameters that are monitored from the same tissue volume, the better and more accurate an understanding of the functional state of the tissue that may be obtained.

There are several techniques that relate to the simultaneous in-vivo measuring of multiple parameters in certain tissues, which can be used for the various pathological situations arising in modem medicine.

The prior art teaches a wide variety of apparatuses/devices which monitor various parameters reflecting the viability of the tissue, for example, in U.S. Pat. No. 4,703,758 and U.S. Pat. No. 4,945,896.

A particular drawback encountered in NADH measurements is the Haemodynamic Artifact. This refers to an artifact in which NADH fluorescence measurements in-vivo are underestimated or overestimated due to the haemoglobin present in blood circulation, which absorbs radiation at the same wavelengths as NADH, and therefore interferes with the ability of the light to reach the NADH molecules. The haemoglobin also partially absorbs the NADH fluorescence. In particular, a reduction of haemoglobin in blood circulation causes an increase in fluorescence, generating a false indication of the true oxidation reduction state of the organ. U.S. Pat. No. 4,449,535 teaches, as means to compensate for this artifact, the monitoring of the concentration of red blood cells, by illuminating at a red wavelength (805 nm) simultaneously and in the same spot as the UV radiation required for NADH excitation and measuring the variation in intensity of the reflected red radiation, as well as the fluorescence at 440-480 nm, the former being representative of the intra-tissue concentration of red blood cells. Similarly Kobayashi et al (Kobayashi et al, 1971) used ultraviolet (UV) illumination at 366 nm for NADH excitation, and red light at 720 nm for reflection measurements. However, U.S. Pat. No. 4,449,535 has at least two major drawbacks; firstly, and as acknowledged therein, using a single optical fiber to illuminate the organ, as well as to receive emissions therefrom causes interference between the outgoing and incoming signals, and certain solutions with different degrees of effectiveness are proposed. Additionally since the same optical fiber is utilised for transmission of excitation light and for transmission of the collected light the excitation and the collection point is the same one. This imposes relatively low penetration depth as can be learned from the paper of Jakobsson and Nilsson (Jakobsson and Nilsson, 1991). More importantly, though, two different wavelengths are used for illuminating the organ. FIG. 2 illustrates the penetration depth profile for various tissues of the human brain as a function of illuminating radiation wavelength, showing a plateau of relative insensitivity of penetration depth (PD) with wavelength, for a wavelength range between about 360 nm and about 440 nm. For illuminating wavelengths greater than 440 nm, the penetration depth increases sharply with wavelength. Similar characteristics are found with other organs of the body. Thus, as may be seen from FIG. 2, the use of light radiation at the red end of the spectrum in accordance with U.S. Pat. No. 4,449,535 or as proposed by Kobayashi, to correct for blood haemodynamic artifacts in the NADH signal introduces inaccuracies into the measurements due to differences in penetration depths and therefore in the actual sampling volumes. Even though both radiation wavelengths are incident on the same spot, since detection is also at the same point, effectively two different elements of tissue volume are being probed since the different radiation wavelengths penetrate the tissue to different depths. This results in measurements that are incompatible one with the other, the blood volume measurement relating to a greater depth of tissue than the NADH measurement. Therefore, the device disclosed by this reference does not enable adequate compensation of NADH to be effected using the simultaneous, though inappropriate, blood volume measurement. There is in fact no recognition of this problem, much less so any disclosure or suggestion on how to solve it. Further, there is no indication of how to measure other parameters such as blood flow rate or blood oxygenation level using the claimed apparatus.

None of these prior art documents suggest monitoring Fp level, with or without any of the other parameters, and less so in an integrated apparatus.

In two earlier patents which have a common inventor with the present invention, U.S. Pat. No. 5,916,171 and U.S. Pat. No. 5,685,313, the contents of which are incorporated herein in their entirety, a device is described that is directed to the monitoring of microcirculatory blood flow (MBF), the mitochondrial redox state (NADH fluorescence) and the microcirculatory blood volume (MBV), using a single source multi-detector electro-optical, fiber-optic probe device for monitoring various tissue characteristics to assess tissue vitality. During monitoring, the device is attached to the fore-mentioned tissue. The probe/tissue configuration enables front-face fluorometry/photometry.

Although U.S. Pat. No. 5,916,171 and U.S. Pat. No. 5,685,313 represent an improvement over the prior art, they nevertheless have some drawbacks:
(i) The oxidation level of the blood will introduce artifacts, affecting both the Mitochrondrial Redox State measurement (NADH fluorescence) and the microcirculatory blood volume (MBV) since these patents do not specify how to compensate for the oxygenation state of the blood in the tissue, i.e., the relative quantities of oxygenated blood to deoxygenated blood in the tissue. As disclosed in Israel Patent Application No. 138683 filed by Applicants, this problem may be overcome by performing the NADH and blood volume measurements at an isosbestic point of the oxyhaemoglobin—deoxyhaemoglobin absorption spectrum.
(ii) There is no facility included for measurement of the oxyhaemoglobin—deoxyhaemoglobin level, i.e. the Blood Oxygenation State, which is also an important tissue viability parameter, worthy of monitoring.
(iii) In these two US patents, the same tissue volume needs to be monitored for all parameters, and the same light source and wavelength is used for the illumination needed for monitoring all three parameters. To measure both the NADH level and the blood flow rate, a relatively powerful UV laser is needed having an illuminating wavelength close to the peak of the NADH excitation spectrum. Using a relatively high intensity UV laser illumination source as proposed raises safety issues, especially for long-term monitoring. An additional problem of NADH photo-bleaching arises since a high intensity UV laser is used.
(iv) The blood flow measurements impose several requirements on the UV laser source. In particular, the UV laser needs to have a high coherence length and very low optical intensity noise. As discussed in more depth below such lasers at these wavelengths have intrinsic properties which tend to discourage their use in such a device, and are in any case quite rare to come by in the first place.
(iv) There is no suggestion of monitoring Fp level, with or without any of the other parameters.

Israel Patent Application No. 138683 filed by Applicants, the contents of which application are incorporated herein in their entirety, further addresses some of these problems by using two separate illumination radiation sources, one for determination of blood flow rate, and the other for determination of at least one tissue viability parameter such as NADH, blood volume and blood oxygenation state. By separating the light sources, the problem of having a single source capable of satisfactorily enabling the determination of blood flow rate as well as the other three tissue vitality parameters is avoided.

While U.S. Pat. No. 5,916,171 and U.S. Pat. No. 5,685,313 ostensibly teach a single illumination radiation for laser Doppler flowmetry and NADH monitoring in the substantially identical tissue volume, on closer scrutiny it is not at all obvious for a man of the art to do so at illuminating wavelengths within the range of between about 370 nm to about 400 nm. There is also absolutely no suggestion whatsoever that the illuminating wavelength should be within the Fp excitation range, i.e., about 400 nm to about 470 nm, and in fact these references teach away from this, as NADH cannot be monitored at all at the higher wavelengths. These patents exemplify a radiation source generating electromagnetic radiation at a wavelength at 366 nm or 325 nm. The reason is twofold. On the one hand, and as illustrated in FIG. 1, the NADH excitation spectrum exhibits a peak near these wavelengths, and therefore illumination of the tissue at any one of these two wavelengths provides sufficient excitation energy to the tissue under investigation, such that the energy of the NADH fluorescence thereby generated is correspondingly high, maximising the sensitivity of measurements. At higher wavelengths, between 370 nm and 400 nm, the NADH excitation spectrum provides sharply diminished excitation intensities, and a man of ordinary skill in the art would thus not normally be motivated to use a radiation source operating at these wavelengths, since the fluorescent radiation from the tissue would effectively be of corresponding low intensity, and therefore difficult to measure accurately. As mentioned earlier, there would be even less motivation to use wavelengths between 400 nm and about 470 nm, and in fact these patents teach away from the same being beyond the NADH excitation spectrum. Also, a man of ordinary skill in the art would not have considered setting the illuminating wavelength at the low intensity-high wavelength shoulder rather than close to the high intensity-low wavelength peak of the NADH excitation spectrum (and much less so, go beyond it). The reason is that there is a possibility of the existence of a second or more excitation spectra similar to that of the NADH, but shifted slightly towards the higher wavelengths, arising due to other components in the blood or tissue which also exhibit a similar excitation spectrum and a similar emission spectrum. Such a second excitation spectrum could interfere with and thus introduce errors in the NADH measurement. (While the present inventors have in fact determined that in practice there are no such second excitation spectra, this would not be known to a man of the art, and the suspicion would remain.)

Furthermore, at the time when these US patents were filed, and indeed until very recently, there were no suitable lasers available capable of generating electromagnetic energy in the wavelength range 370 nm to 400 nm, or indeed in the range 400 nm to about 470 nm with sufficiently low Relative Intensity Noise factor (RIN). The two lasers that were then available were a 325 nm Helium Cadmium laser and a 355 nm “3^{rd} Harmonic of Nd-Yag” laser. The He-Cd laser is a large gas laser, having relatively large power consumption, being generally unsuitable for the applications where small size and power consumption are important considerations. Furthermore, this laser generates a great deal of optical noise, having a Relative Intensity Noise factor (RIN) of about 1% to 2%. There is also a small but significant spectral spread at the operating wavelength, typically comprising about eleven discrete wavebands bundled thereabout, further diminishing the efficiency of operation. While this laser enables single illumination radiation for laser Doppler flowmetry and NADH monitoring, the sensitivity is very low, and operation of such a laser raises many safety issues, since operating at a wavelength of 325 nm carries potential risk of DNA damage to the tissue. The Nd-Yag laser provides radiation at a higher wavelength of 355 nm. However, it generates a great deal of optical noise when operating in continuous wave (CW), resulting in poor quality measurements. While this laser generates less noise in pulse mode, no useful measurements may be made for Doppler Flowmetry using pulsed lasers, since it is very difficult to ensure uniformity between the pulses generated.

Furthermore, there would be little motivation for a man of the art to use a laser at the illuminating wavelength range of 370 nm-400 nm, or indeed in the range of about 400 nm to about 470 nm for laser Doppler Flowmetry, even if one existed, for a number of reasons. Laser Doppler Flowmetry as applied to a tissue volume substantially comprising capillarial blood flow is substantially different from the laminar flow Laser Doppler flowmetry methods used with large tissues having veins or arteries.

In the laminar flow laser Doppler flowmetry the laser ray is split and then converged again in order to produce interference fringes in the path of fluid flow. As fluid particles pass through these fringes they produce an alternating light signal, which can be analysed to provide a measure of particle velocity and fluid flow rate. In such a method, severe constraints are imposed on the laser spectral bandwidth that is acceptable for the task. Broad laser bandwidth causes blurring of the interference fringes, thereby decreasing the quality of the measurements.

In contrast, the laser Doppler blood flowmetry method used in the present invention relates to the measurement of blood perfusion through tiny capillaries. The flow is not laminar but random in velocity and direction. The illumination of the tissue is done through a single optical fiber and the collection performed by at least one collection fiber. The most intuitive way to understand the blood laser Doppler measurements is by specklemetery. The laser light is shone onto a tissue containing the random network of tiny capillaries through which the Red Blood Cells (RBC) are flowing. Both the direction and velocity of the RBC flow are random. As the laser radiation penetrates the tissue bulk, some small part of the excitation light is reflected after numerous scatterings inside the tissue. This reflected light produces a random interference pattern referred to as a speckle pattern on the tissue surface. The RBC movements inside the capillaries cause random changes in this speckle pattern. The collection optical fiber, which is placed near to the excitation fiber, delivers to an appropriate detector the changes in speckle intensity due to the blood flow, and an electrical signal corresponding to the changing intensity is generated. The Doppler signal is thus represented by small fluctuations of the total light intensity, i.e. the AC ripple in relation to the DC total intensity, which represents the reflection signal that is correlated to blood volume. From analysis of the detector power spectrum the blood flow parameter can be deduced according to the algorithm described by Bonner and Nossal [Bonner and Nossal, 1990]. Thus, the flowmetry method is based on measuring the perturbations in the intensity of the electromagnetic radiation received from the tissue in relation to the mean intensity of the radiation, in other words, the ratio between the AC signal to the DC signal of the radiation received by the monitoring probe. With lasers operating at large wavelengths, for example red lasers, the penetration of the laser into the tissue is relatively large, and therefore the AC signal is proportionally larger, since more capillaries interact with the illuminating laser radiation. In other words, as the laser wavelength increases, or as the corresponding laser bandwidth narrows, the speckle pattern becomes more defined and the intensity fluctuations became higher. It follows that with lasers operating at lower wavelengths the opposite is true, and at lower illuminating radiation wavelengths, including the range 370 nm to 470, and in particular in the range 370 nm to 400 nm, the penetration of the laser radiation into the tissue would be less, providing a lower AC signal relative to the mean DC signal, which drastically lowers sensitivity.

However, there are further problems associated with using an illuminating radiation wavelength in the range 370 nm to 470 nm that teach away from using such a laser wavelength for Doppler flowmetry:—
(a) Firstly, the magnitude of the actual DC signal is lower than with higher-wavelength lasers because of higher tissue and blood absorption as well as higher scattering, which therefore results in lower sensitivity in the measurement of the AC/DC ratio.
(b) Secondly, safety issues are raised with using such a laser wavelength range, as described in greater detail hereinbelow.
(c) Thirdly, the optical noise generated by the laser, while not a severe problem with high-wavelength lasers, in UV lasers this can be of the same order as the Doppler signal itself, thereby obscuring the parameter being measured.
(d) Fourthly, detectors capable of detecting the AC component of the radiation received from the tissue are not generally very sensitive in the wavelength range 370 nm to about 470 nm, which of course lowers further still the chances of successfully using Doppler flowmetry at this wavelength range.
(e) Fifthly, laser Doppler flowmetry as applied to the determination of capillarial blood flow rate relies on detection and measurement of a refracted laser speckle pattern produced at the surface of the tissue on which the laser is emitting the illuminating radiation. At low illuminating wavelengths, the speckle pattern is considerably smaller than at higher wavelengths, which lowers the possibility even further of such speckles being detected and measured in the first place.
(f) Sixthly, the optical fibers that transmit the optical signals to and from the tissue are not as optically efficient in the 370 nm to 470 nm range as they are at the higher wavelengths.

All the above problems individually, and more so in combination, teach away from considering the use of a laser in the 370 nm to 470 nm range for measuring blood flow rate together with blood NADH or with Fp, since the combined inefficiencies reduce the possibility of providing meaningful flowmetry results. However, even if the problem of decreased sensitivity is resolved, there are yet another two problems that dissuade the use of such lasers in the present context.

Firstly, it is by far most convenient to provide an illuminating radiation at an isobestic wavelength to mimimise the effect of the Haemodynamic Artifact on measurements. For NADH fluorescence, an isosbestic wavelength in the range 370 nm to 400 nm exists at a wavelength of about 390 nm. Similarly, an isosbestic point exists in the Fp excitation spectrum between about 400 nm and 470 nm wavelengths, at about 455 nm. Until recently, no laser diodes capable of operating at these isosbestic wavelengths were available. Recently, though, a range of laser diodes by Nichia Chemical Industries Ltd., Anan, Japan capable of operating in continuous mode within the range 385 nm to 440 nm has become available, including the violet laser diode such as the NLHV500. However, even these lasers are still subject to the above problems.

Secondly, even the existence of such a laser in and of itself does not render its use obvious in the context of laser Doppler flowmetry and NADH monitoring. For example, if such a laser were to be used in combination with the device disclosed in U.S. Pat. No. 5,916,171 and U.S. Pat. No. 5,685,313, the device would still be incapable of providing meaningful Doppler flowmetry measurements. The reason for this is as follows. While lasers generate electromagnetic radiation nominally at a single wavelength, in practice, this is not achieved, and two or more discrete narrow wavebands are generated. This plurality of wavebands are herein referred to as longitudinal multi-mode radiation, and is different conceptually and practically to the “transverse multi-mode” radiation commonly encountered with many lasers. The phenomenon of longitudinal multi-mode radiation generation occurs as a result of more than one stable wavelength being generated by the laser in general and also the laser diode itself due to the physical constraints imposed by the laser cavity. While very close in wavelength, these wavebands are nonetheless discrete. For example, in the NHLV500 diode which operates nominally at 405 nm wavelength has several longitudinal modes separated by about 0.05 nm or 95 GHz, as illustrated in FIG. 5. The typical bandwidth of each such mode is rather broad, in the order of 400 MHz. The effect of the longitudinal multi-mode illumination radiation is to provide a speckle pattern on the tissue of low contrast as compared with a longitudinal single mode illuminating radiation, that is comprising a single waveband, which degrades the flowmetry measurements. Also, the corresponding RIN is higher than with single mode radiation, such that with more than about two bandwidths, the optical noise associated with multi-mode radiation is of the same order as the AC levels being monitored, such as to render such measurements substantially meaningless. Thus, longitudinal multimode operation under conditions of relatively high optical noise and broad bandwidth is not suitable for laser Doppler measurements.

A further problem associated with longitudinal multi-mode radiation is the phenomenon of mode competition, in which the actual wavelength of the illuminating radiation randomly switches from one of the discrete modes or wavebands to another, which dramatically increases the level of RIN. Thus, for good flowmetry measurements a very low noise laser with preferably a single longitudinal mode and as narrow as possible bandwidth associated therewith is needed, and thus there is no motivation for a man of the art to combine off-the-shelf lasers of wavelength between about 370 nm to about 400 nm with the device of U.S. Pat. No. 5,916,171 or U.S. Pat. No. 5,685,313. As mentioned above, there is even less motivation for a man of the art to combine lasers of wavelength 400 nm to 470 nm with the device of these patents. Finally, even if such a laser diode were to be configured to generate radiation in nearly longitudinal single mode by critical choice of current and temperature, which is in itself far from self-evident, factors such as temperature and current drifts may cause regression to multi-mode operation. Furthermore such single mode operation still has intrinsically a very broad bandwidth in order of 400 Mhz, which of itself is still problematic for laser Doppler flowmetry. Thus, all the above factors would tend to teach away from employing such a laser configuration for multiparamater monitoring In fact, even a grating-stabilized laser diode that nominally operates in a single longitudinal mode exhibits intensity instability. Generally, this happens when, during operation, the operating parameters of the laser system change due to aging thermal drift etc. Due to such changes the laser system gradually drifts to highly unstable multimode operation accompanied with very high optical noise caused by mode competition.

In any case, apparatuses that incorporate a laser light source are generally required to comply with relevant laser safety standards. The two relevant standards which deal with exposure of human tissue to laser radiation are the ANSI Z136.1-2000 “American National Standard for Safe Use of Lasers” and the IEC60825-1-1994 International Standard called “Safety of laser products”.

These standards define the Maximum Permissible Exposure (MPE) values. These standards relate to laser irradiation of external tissues such as skin and eye and not of the internal organs, in contrast to typical applications of the present invention. Still they are the only known, well established references to safe irradiation values for tissues, and any laser device that is intended to perform nondestructive measurements should comply with these in the absence of a more appropriate full damage test being performed on specific tissue type with specific light irradiation.

Both the above standards permit a maximum of 1 mW/cm² irradiance for exposure time larger then 1000 sec. This requirement implies a severe limitation on the light intensity emitted by the distal tip of the fiber optic probe, particularly when shorter wavelength, higher intensity radiation is used. These values correspond to laser wavelengths in the range of between about 315 nm to about 400 nm. At higher wavelengths than about 400 nm, the (MPE) values are higher.

It is an aim of the present invention to overcome the above deficiencies in the prior art.

Particularly, it is an aim of the present invention to provide an apparatus enabling the simultaneous in-vivo monitoring of blood flow rate (i.e. intravascular mean velocity times the number of moving red blood cells) and at least one, and preferably all, of the following set: Mitrochondrial Redox State via NADH concentration by fluorescence, total blood volume (i.e. concentration of red blood corpuscles) by reflectometry, blood haemoglobin oxygenation (i.e. the oxy/deoxy haemoglobin ratio) by NADH fluorescence; or alternatively blood flow rate and at least one of and preferably all of Mitrochondrial Redox State via flavoprotein concentration by fluorescence, optionally including total blood volume by reflectometry and/or blood haemoglobin oxygenation by fluorescence, based on flavoprotein fluorescence; for the same body tissue, in substantially the same tissue element. These parameters, which represent different biochemical and physiological activities of the tissue, are used to assess the tissue vitality in said tissue element.

It is another aim of the present invention to provide a device or apparatus capable of monitoring blood flow rate and at least one other tissue vitality parameter including NADH level, in a substantially identical tissue volume, using a single illuminating radiation having a wavelength in the range from about 370 nm to about 400 nm, and more particularly at about 390 nm.

It is another aim of the present invention to provide a device or apparatus capable of monitoring blood flow rate, NADH level, blood volume and blood oxygenation state in a substantially identical tissue volume, using a single illuminating radiation having a wavelength in the range from about 370 nm to about 400 nm, and more particularly at about 390 nm.

It is another aim of the present invention to provide a device of apparatus capable of monitoring blood flow rate and at least one other tissue vitality parameter including flavoprotein level, in a substantially identical tissue volume, using a single illuminating radiation having a wavelength in the range from about 400 nm to about 470 nm, and more particularly at about 440 nm or 455 nm.

It is another aim of the present invention to provide a device or apparatus capable of monitoring blood flow rate, flavoprotein level, blood volume and blood oxygenation state in a substantially identical tissue volume, using a single illuminating radiation having a wavelength in the range from about 400 nm to about 470 nm, and more particularly at about 440 nm or 455 nm.

It is another aim of the present invention to provide such a device or apparatus that provides a single illuminating radiation at longitudinal single mode having a single bandwidth.

It is another aim of the present invention to provide such a device or apparatus that provides a single illuminating radiation at longitudinal multi-mode comprising three or less bandwidths in non-competing modes.

It is another aim of the present invention to provide such a device or apparatus that provides for the stabilisation of such a longitudinal single mode or such a longitudinal non-competing multi-mode.

It is another aim of the present invention to provide such a device or apparatus that conforms to the relevant laser safety standards.

It is another aim of the present invention to provide such a device or apparatus that is of a convenient size, weight and power consumption such as to enable the same to be portable and/or installable within regular operating theaters.

These and other aims are achieved in the present invention by providing a device or apparatus capable of generating an illuminating laser radiation characterised in one embodiment in having a nominal wavelength in the range of about 370 nm to about 400 nm, and in particular about 390 nm, adapted for monitoring NADH level, blood volume and blood oxygenation state as well as blood flow rate, and in another embodiment in having a nominal wavelength in the range of about 400 nm to about 470 nm, and in particular about 440 nm and more particularly 455 nm, adapted for monitoring flavoprotein level, blood volume and blood oxygenation state as well as blood flow rate. The invention is further characterised in providing means to filter out most of unwanted bandwidths generated naturally by the laser, and thus provide a longitudinal single mode illuminating radiation to the tissue, or a typically two or three non-competing multi-mode illuminating radiation to the tissue, such as to enable blood flow rate and at least one of, and preferably all of the set comprising NADH level, blood volume and blood oxygenation state or at least one of and preferably all of the set comprising flavoprotein level, blood volume and blood oxygenation state to be determined for the substantially identical tissue volume. The invention further provides for the stabilisation of the illuminating radiation wavelength, such as to prevent regression to a competing multi-mode situation. Typically one or a bundle of optical fibers are provided for illuminating the tissue at nominally a single location thereon, together with one or bundle of detection fibers. The detection fibers are all substantially equi-distant from the illuminating fibers, thereby ensuring that the substantially identical tissue volume is the subject of all the measurements.

Other purposes and advantages of the invention will appear as the description proceeds.

### SUMMARY OF THE INVENTION

The present invention relates to an apparatus for selectively monitoring a blood flow rate tissue viability parameter and at least one second tissue viability parameter corresponding to a substantially identical tissue element, the apparatus comprising:—
illumination means for illuminating at least said tissue element with an illuminating radiation via at least one illumination location with respect to said tissue element;
radiation receiving means for receiving a radiation from said tissue element as a result of an interaction between said illuminating radiation and said tissue element, wherein a part of said received radiation is correlated to said blood flow rate tissue viability parameter, and wherein another part of said received radiation is correlated to said at least one second tissue viability parameter, said radiation receiving means being displaced from said illumination location by a first displacement;
characterised in that said illuminating radiation is a laser radiation having a nominal wavelength in the range from about 370 nm to about 470 nm.

In preferred embodiments, the laser radiation is generated in stable single longitudinal mode, wherein said nominal wavelength comprises a single waveband element, and the waveband element typically comprises a bandwidth of about 4 MHz.

In other embodiments, the said laser radiation is generated in two or three stable longitudinal non-competing modes, wherein said nominal wavelength comprises two or three, respectively, discrete waveband elements. The waveband elements each typically comprise a bandwidth of about 4 MHz.

The illumination location is provided by at least one excitation optical fiber having a free end capable of being brought into registry with said tissue element. The radiation receiving means comprises at least one suitable receiving optical fiber having a free end capable of being brought into registry with said tissue element. The at least one excitation optical fiber and said at least one receiving optical fiber are preferably housed in a suitable probe head, wherein said free end of said at least one excitation fiber and said free end of said at least one receiving fiber are comprised on a contact face of said probe. Preferably, the at least one excitation fiber comprises a suitable first connector at an end thereof opposed to said free end thereof, said first connector capable of selectively coupling and decoupling said excitation fiber from the rest of the said apparatus. Similarly, the at least one collection fiber also preferably comprises a suitable second connector at an end thereof opposed to said free end thereof, said second connector capable of selectively coupling and decoupling said collection fiber from the rest of the said apparatus. The probe may be disposable and/or sterilisable.

In preferred embodiments, the illumination means comprises a suitable external cavity laser diode system, typically based on a suitable violet laser diode having an operating wavelength in the range of between about 370 nm and about 470 nm. The external cavity laser diode system may be configured according to the Littrow design or according to the Metcalf-Littman design. Preferably, the external cavity diode laser system comprises a laser stabilisation control system for ensuring stable single mode operation of the said external cavity laser diode system. Typically, the laser stabilisation control system is adapted for monitoring the laser intensity of the said external cavity laser diode system at a predetermined input current to said external cavity laser diode system and providing an electrical signal representative of said intensity, for varying the said input current within a predetermined range to provide corresponding electrical signals correlated to the resulting laser intensities generated, for identifying the corresponding electrical signal providing minimum RIN noise levels, and for adjusting the said input current such as to provide and maintain said electrical signal providing minimum RIN noise levels.

In preferred embodiments, the blood flow rate tissue viability parameter is provided by applying a laser Doppler flowmetry technique to said radiation received by said radiation receiving means, and the apparatus further comprises first detection means for detecting said received radiation received by said radiation receiving means.

Preferably, the illumination means is adapted to provide said illuminating radiation in pulses of predetermined duration and intensity by correspondingly chopping the illuminating radiation generated by said illuminating means. The apparatus further comprises suitable control means for controlling the frequency of pulsing of said pulses. The control means may be further adapted to provide said pulses in packages of pulses, each package comprising at least one pulse and separated from a preceding or following package by a predetermined time period. The predetermined time period may be greater than the time interval between consecutive pulses within a package, and the time period and/or the number of pulses within each package may be controllably variable.

Preferably, the control means is operatively connected to said first detection means.

In the first preferred embodiment, the nominal wavelength is at wavelength within the NADH excitation spectrum, preferably at a suitable isobestic wavelength within the NADH excitation spectrum, and more preferably is about 390 nm±5 nm.

In this embodiment, one of the second tissue viability parameters is NADH concentration, wherein said radiation received by said radiation receiving means comprises an NADH fluorescence emitted by the tissue in response to illumination thereof by said illuminating radiation, said at least one second tissue viability parameter being provided by the intensity of said NADH fluorescence. The apparatus thus may comprise suitable second detection means for detecting said received radiation received by said radiation receiving means. Further, the control means may be operatively connected to said second detection means, and the control means may be selectively responsive to previously detected signals corresponding to the detection of said received radiation detected by means of said second detection means of a prior monitoring cycle.

In the second preferred embodiment, the nominal wavelength is at wavelength within the Fp excitation spectrum, preferably at a suitable isobestic wavelength within the Fp excitation spectrum, and more preferably is about 440 nm±5 nm.

In this embodiment, one of the second tissue viability parameters is Fp concentration, wherein said radiation received by said radiation receiving means comprises an Fp fluorescence emitted by the tissue in response to illumination thereof by said illuminating radiation, said at least one second tissue viability parameter being provided by the intensity of said Fp fluorescence. The apparatus thus may comprise suitable second detection means for detecting said received radiation received by said radiation receiving means. Further, the control means may be operatively connected to said second detection means, and the control means may be selectively responsive to previously detected signals corresponding to the detection of said received radiation detected by means of said second detection means of a prior monitoring cycle.

The first and second preferred embodiments may, in addition or in lieu of NADH or Fp concentration, respectively, include monitoring of another second tissue viability parameter, namely blood volume within said tissue element, and in this case said corresponding radiation received by said radiation receiving means comprises a reflection from the tissue element in response to illumination thereof by said illuminating radiation, the said at least one second tissue viability parameter being provided by the intensity of said reflection. Thus, the apparatus further comprises third detection means for detecting said received radiation received by said radiation receiving means. The control means may be operatively connected to said third detection means, and the control means may be selectively responsive to previously detected signals corresponding to the detection of said received radiation detected by means of said third detection means of a prior monitoring cycle.

Similarly, another second tissue viability parameter may be monitored, in addition to or in lieu of either to the other parameters, i.e., blood oxygenation ratio within said tissue element, and said corresponding radiation received by said radiation receiving means is a fluorescence emitted by the tissue in response to illumination thereof by said illuminating radiation, said at least one second tissue viability parameter being provided by the intensity of said fluorescence at least at two fluorescent emission wavelengths.

For the first preferred embodiment, one of said at least two fluorescent wavelengths is chosen to lie at an isosbestic point of the NADH fluorescence emission spectrum. Alternatively, and preferably, one of said at least two fluorescent wavelengths is higher and another one of said at least two fluorescent wavelengths is smaller than a wavelength corresponding to an isosbestic point of the NADH fluorescence emission spectrum. Preferably, blood oxygenation ratio parameter is provided by normalising said fluorescent intensities at said two wavelengths with respect to the fluorescent emission intensity at said isosbestic point of said NADH fluorescence emission spectrum. The wavelength corresponding to one such isosbestic point is about 455 mm±5 nm. Preferably, the apparatus further comprises fourth detection means for detecting said received radiation received by said radiation receiving means. The control means is operatively connected to said fourth detection means, and the control means is selectively responsive to previously detected signals corresponding to the detection of said received radiation detected by means of said fourth detection means of a prior monitoring cycle. For the second preferred embodiment, one of said at least two fluorescent wavelengths is chosen to lie at an isosbestic point of the Fp fluorescence emission spectrum. Alternatively, and preferably, one of said at least two fluorescent wavelengths is higher and another one of said at least two fluorescent wavelengths is smaller than a wavelength corresponding to an isosbestic point of the Fp fluorescence emission spectrum. Preferably, blood oxygenation ratio parameter is provided by normalising said fluorescent intensities at said two wavelengths with respect to the fluorescent emission intensity at said isosbestic point of said Fp fluorescence emission spectrum. The wavelength corresponding to one such isosbestic point is about 530 nm±5 nm. Preferably, the apparatus further comprises fourth detection means for detecting said received radiation received by said radiation receiving means. The control means is operatively connected to said fourth detection means, and the control means is selectively responsive to previously detected signals corresponding to the detection of said received radiation detected by means of said fourth detection means of a prior monitoring cycle. The present invention is also directed to a system for selectively monitoring at least two tissue viability parameter at a plurality of tissue elements. The system comprises a plurality of monitoring probes, each said probe being substantially similar to that comprised in the apparatus according to the first or second embodiments.

In the system, at least two said probes are adapted for monitoring said tissue viability parameters of tissue elements within the same organ. Alternatively, at least two said probes are adapted for monitoring said tissue viability parameters of tissue elements within different organs, wherein different organs are different organs within the same organism, or wherein different organs are different organs within different organisms. The different organs may comprise include donor organs.

The illuminating radiation for each said probe may be provided by a common suitable light source, which is a laser light source as in the first and second embodiments.

The laser light source may be adapted to provide said first illuminating radiation of said first wavelength in second pulses of predetermined duration and intensity. The system may further comprise suitable control means for controlling the frequency of pulsing of said second pulses. The control means may be further adapted to provide said second pulses in packages of pulses, each package comprising at least one second pulse and separated from a preceding or following package by a predetermined time period the predetermined time period may be greater than the time interval between consecutive pulses within a package. Preferably, the time period may be controllably variable, and the number of second pulses within each package may be controllably variable. The control means may be adapted for selectively directing discrete said second pulses to any one of said probes.

### BRIEF DESCRIPTION OF THE DRAWINGS

The present invention will be more clearly understood from the detailed description of the preferred embodiments and from the attached drawings in which:

FIG. 1 shows the excitation fluorescence spectrum (F_{EXT}) and emission fluorescence spectrum (F_{EMS}) for NADH, in terms of the corresponding fluorescence intensities (IF) as a function of wavelength (WL).

FIG. 2 illustrates typical penetration depth characteristics for human brain tissues as a function of illumination wavelength.

FIG. 3 illustrates light absorption of blood oxy-haemoglobin and blood deoxyhaemoglobin in terms of an Extinction parameter (E) as a function of wavelength (WL).

FIG. 4 illustrates schematically the main components of the first and second preferred embodiment according to the present invention.

FIG. 5 illustrates the multimode characteristics of a laser radiation at nominal wavelength of about 405 nm for a Nichia violet laser diode, in terms of relative intensity (RI) as a function of wavelength (λ).

FIG. 6(a) illustrates schematically the main components of an external cavity diode laser system according to the Littrow design.

FIG. 6(a) illustrates schematically the main components of an external cavity diode laser system according to the Metcalf-Littman design.

FIG. 7 illustrates various factors that determine the exact laser output frequency of the external cavity laser diode system.

FIG. 8(a) illustrates, in transverse cross-sectional view, a probe according to a preferred embodiment of the present invention.

FIG. 8(b) illustrates in end view the embodiment of FIG. 8(a) taken along X-X.

FIG. 9(a) illustrates schematically the fluorescence intensity (IF) emitted by a tissue as a function of wavelength (WL) and oxygenation level of the blood contained in the tissue.

FIG. 9(b) illustrates schematically the ratio of fluorescence intensities at two wavelengths with respect to the fluorescence intensity at an isosbestic point of FIG. 9(a).

FIG. 10(a) schematically illustrates a circuit diagrams for a signal detector optionally used with the embodiment of FIG. 4.

FIG. 10(b) schematically illustrates a circuit diagrams for another signal detector optionally used with the embodiment of FIG. 4.

FIG. 10(c) schematically illustrates a circuit diagrams for another signal detector optionally used with the embodiment of FIG. 4.

FIG. 11(a) shows the main clock sequence that enables the transmission of the light from light source (101) by the acousto-optic modulator (AOM).

FIG. 11(b) shows the output voltage of the detector in response to the light modulated by the AOM.

FIG. 11(c) shows the clock sequence applied to the sample and hold (S/H) circuitry (440) as shown in FIG. 10.

FIG. 11(d) shows the light signal as it appears at the output of (S/H) circuitry (440) of FIG. 10.

FIG. 11(e) shows the sequence train of pulses as provided during state II operation of the device.

FIG. 11(f) shows the sequence train of pulses as provided during state III operation of the device.

FIG. 12 shows the excitation fluorescence spectrum (Fp_{EXT}) and emission fluorescence spectrum (Fp_{EMS}) for Fp, in terms of the corresponding fluorescence intensities (IF) as a function of wavelength (WL).

FIG. 13 illustrates schematically the main components of the third preferred embodiment according to the present invention.

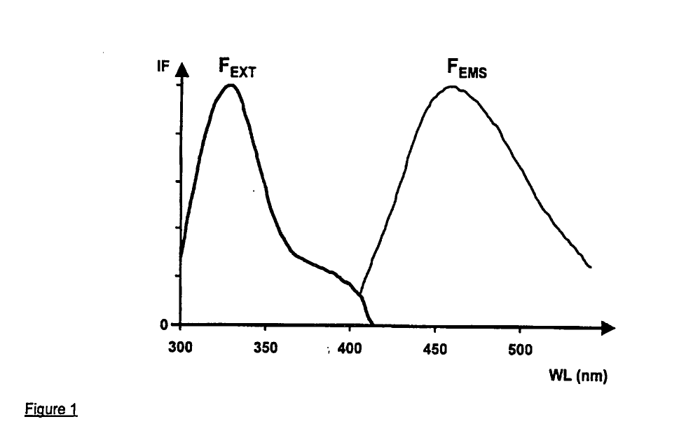

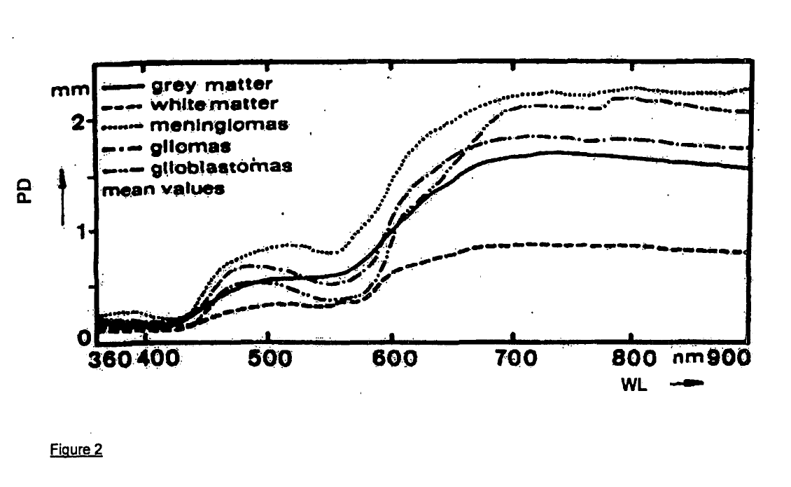

### DETAILED DESCRIPTION OF PREFERRED EMBODIMENTS

The present invention is defined by the claims, the contents of which are to be read as included within the disclosure of the specification, and will now be described by way of example with reference to the accompanying figures.

In the description to follow, the following illustrative apparatuses and methods are described, it being understood that the invention is not limited to any particular form thereof, and the following description being provided only for the purposes of illustration.

The present invention is directed to an apparatus for simultaneously monitoring at least two tissue viability parameters from a substantially identical volume of tissue element. In particular, one of these parameters is the blood flow rate corresponding to the tissue volume, and the other tissue viability parameter includes at least one of, and preferably more than one of, and most preferably all of, the set of parameters comprising at least NADH concentration or flavoprotein concentration, blood volume and blood oxygenation state corresponding to the tissue volume. A laser radiation source provides a single illumination radiation at a particular excitation wavelength that is used for monitoring these parameters, as will be described in detail hereinbelow. In particular, means are provided to ensure that the excitation radiation is at a single mode bandwidth, or at least at three or less stable bandwidths, i.e., that are not in competition with each other. Thus the blood flow rate measurement is conducted concurrently with the monitoring of the other tissue viability parameters, providing simplicity in terms of configuration and design of the monitoring apparatus, as well as in the method of use, as will be evident from the following description.

In the present specification, the magnitudes of wavelengths specified herein may be varied by about ±5 nm, and even up to about ±10 nm without significantly affecting operation of the apparatus of the invention.

According to embodiments of the present invention in which the tissue vitality parameters being monitored (other than blood flow rate) are based on the NADH parameter, the excitation wavelength is in the range between about 370 nm and about 400 nm. According to other embodiments of the present invention in which the tissue vitality parameters being monitored (other than blood flow rate) are based on the flavoprotein parameter, the excitation wavelength is in the range between about 400 nm and about 470 nm.

Preferably, an excitation wavelength is chosen such as to simplify correction for the haemodynamic artifact. The haemodynamic artifact arises from the absorption of the NADH fluorescence emission and excitation light by the blood haemoglobin. A change in blood volume cause misleading changes in apparent NADH fluorescence. Since blood haemoglobin has two oxygenation states namely oxy-haemoglobin and deoxy-haemoglobin each one with its distinct absorption spectrum, as shown in FIG. 3, the precise correction of the haemodynamic effect can become extremely complex. The problem is considerably simplified when the wavelength chosen for NADH fluorescence excitation corresponds to one of the isosbestic points, since at these wavelengths the absorption of both haemoglobin species is identical. At these isosbestic excitation points, fluorescence changes are due substantially to changes in total blood volume, and, of course, to changes in NADH concentration only. Thus, by suitably choosing the excitation wavelength, correction for the haemodynamic artifact is significantly simplified. Even at wavelengths within the isosbestic range of from about 300 nm to about 340 nm, or at the isosbestic point of 390 nm, the haemodynamic artifact requires correction. Suitable algorithms for this purpose are described on the prior art (Koyabashi et al., 1971; Renault G., et al. American Journal of Physiology, 246, H491-H499, 1984; Mayevsky A. and Chance B., Brain Res. 65, 529-533, 1974; Harbig et al., J. Appl. Physiol. 41, 480-488, 1976; U.S. Pat. No. 4,449,535). The most widely used correction algorithm (Jobsis et al. Neurophysiology 3465, 735-749, 1971) utilizes the value of the reflection at the NADH excitation wavelength as an indicator for blood changes. The corrected NADH fluorescence values are calculated by subtraction of the reflection signal from the fluorescence signal. In addition, the reflection measurements that provide blood volume correlated signals can also be disturbed by the Haemodynamic Artifact, since the reflection will be influenced not only by the total haemoglobin concentration, but also by the relative concentration of the oxy- and deoxy-haemoglobin. Within the range of excitation wavelengths of the present invention, an isobestic point exists at a wavelength of about 390 nm, and in the preferred embodiment, this is also the target excitation wavelength.

Similar considerations apply to the haemodynamic artifact arising from Fp fluorescence emission and excitation light by the blood haemoglobin, mutatis mutandis.

As is explained in greater detail herein, in the present invention the problems normally associated with using an illuminating radiation wavelength in the range 370 nm to about 470 nm, are addressed as follows.

(a) Low sensitivity in the measurement of the AC/DC ratio and (b) safety issues. In the present invention, a relatively high intensity laser is used together with a chopping technique. This enables high intensity irradiation while mean irradiation intensity is hold below safety limit for the tissue. Additionally, high gain low noise detectors are also employed. (c) Optical noise generated by the laser. In the present invention, a specially stabilized low noise laser is used. (d) Sensitivity to detecting the AC component in the wavelength range 370-470 nm. In the present invention, special UV—Blue enhanced photodiodes with high sensitivity at UV or blue region are used. (e) Difficulty in detecting speckle pattern. In the present invention, thin collection fibers are used in order to enhance sensitivity to intensity fluctuations caused by speckle movement into and out the collection fiber area. (f) Poor optical efficiency in the 370 nm to 470 nm range In the present invention this is overcome by using, for example, silica high OH optical fibers, which have better transmission at UV.

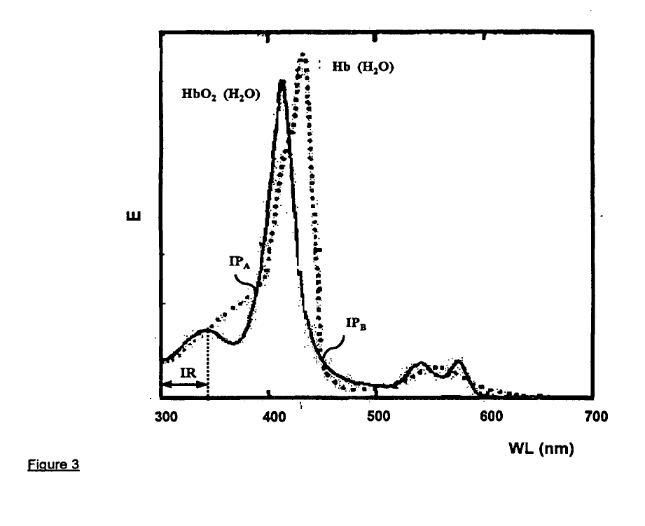

Referring in particular to FIG. 4, in the preferred embodiment of the present invention the laser radiation source is used for laser Doppler flowmetry (LDF), and for monitoring at least one, and preferably all of NADH, blood volume and oxy-deoxy haemoglobin levels. In the present invention, the laser radiation source (101) is comprised in a light source unit (LSU), shown at (1), and is based on a NLHV500 laser diode, manufactured by Nichia Chemical Industries Ltd., Anan, Japan. Other laser diodes that may operate at the required illuminating wavelengths may also be suitable according to availability, mutatis mutandis. However, modification of the diode and/or its operation are required in order to operate successfully in the present invention.

Normally, laser diodes are sold as OEM modules such as PPM(400-5) from Power Technology, Little Rock, Ark., USA. These modules provide laser diode temperature stabilization, laser diode current driver and a collimating lens. Although the experience with laser Doppler flowmeters based on red laser diodes shows that the regular current driver and temperature stabilization provided by such module can be sufficient for Doppler measurements, this is not the situation with the violet laser diodes. In particular, the NLHV500 laser diode with regular current driver and temperature stabilization was found by the present inventors to be problematic for Doppler flowmetry, and therefore in its current form unsuitable for the present invention.

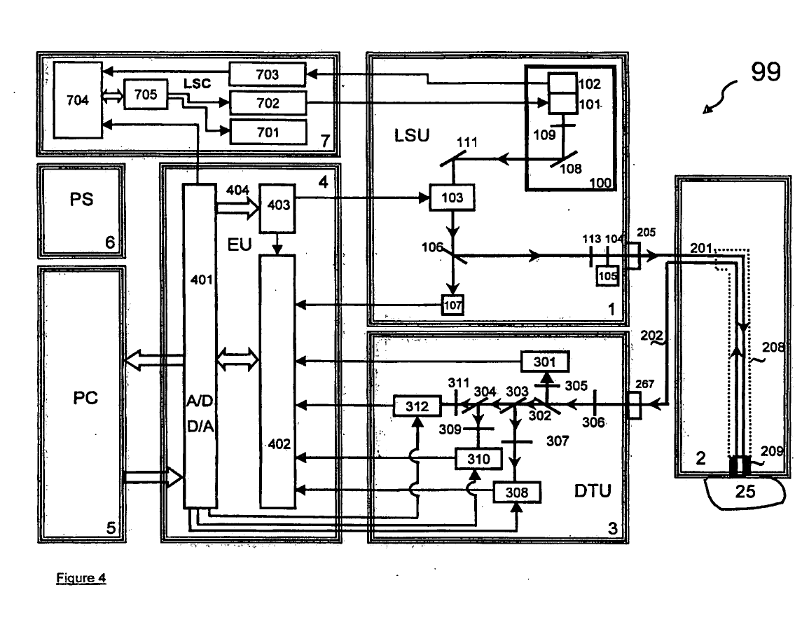

There are two other aspects of the violet laser diode radiation that where also found by the present inventors to be problematic in the context of the present invention, and may be present in other laser diodes of the same operating frequency. The first aspect is that of laser light amplitude noise. The NLHV500 laser diode emits several longitudinal modes as can be seen on FIG. 5. Mutual competition of these modes are expressed in relatively high intensity noise. The second problematic aspect is that of laser diode global bandwidth. The term “global bandwidth” herein refers to the range of wavelengths that includes all the operating modes of the laser diode. In general a free-running laser diode is a high-gain device with very low cavity finesse, and generates a relatively large fundamental bandwidth. Typically a red free-running single longitudinal mode laser diode source emits laser radiation over a bandwidth of 10 to 15 MHz. While this bandwidth is narrow enough for laser Doppler blood flowmetry at for example at 720 nm, for laser diodes that operate at lower wavelengths, such as the NLHV500, the output spectral properties are much worse. The violet laser diode NLHV500, for example, has a longitudinal multi-mode output as shown in FIG. 5. This kind of laser diode has several longitudinal modes separated by about 0.05 nm or 95 GHz while the typical bandwidth of each such mode is very broad in order of 400 Mhz. Therefore even if such a laser diode were to be configured to generate radiation in nearly longitudinal single mode by critical choice of current and temperature, such single mode operation still has intrinsic very broad bandwidth in order of 400 Mhz. Therefore this typical behavior of laser diodes operating at the range of wavelengths 370 nm to 470 nm, is nonetheless unsuitable for laser Doppler flowmetry.

In the present invention, the broad mode bandwidth and global bandwidth obtained with such diodes is effectively and dramatically reduced by utilizing an external cavity system (Nakamura S. and Kaenders W. Market-ready blue diodes excite spectroscopists. Laser Focus World, April 1999). As illustrated in FIG. 6(a), an external cavity laser diode system, (ECLD) (100) such as the DL100 from Toptica Photonics AG, Munich, Germany may be constructed based on the violet laser diode (101), such as the NLHV500 diode. In the ECLD system (100), a collimating lens (109) is provided along the optical axis of the basic laser diode (101), and a special grating (108), inclined at a particular angle and intersecting the optical axis of the diode (101) is used for feeding back part of the laser radiation back to the laser diode. In general, as polychromatic light is illuminated onto such a grating, which comprises a reflecting surface with many grooves, the light will be subsequently reflected towards several directions, called orders. In each order the light of a corresponding different wavelength is reflected towards slightly different direction. Thus, in each order there is some amount of directional separation between the wavelengths. This separation does not exist in what is known as the “zero order”. Typically, the first order has the highest intensity of reflected light. Thus, the laser radiation incident on the grating (108) is spectrally filtered by the grating (108), wherein wavelength of the first order is reflected directly back to the laser diode (101), while the zero order is coupled out.

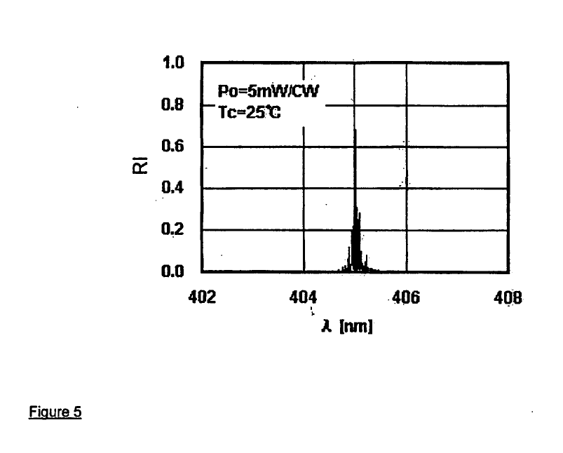

FIG. 7 illustrates various factors that determine the exact laser frequency of the laser radiation generated and outputted by the external cavity laser diode system (100). The medium gain (MG) of the diode (101) is determined by the intrinsic properties of the laser diode chip thereof. The internal mode (IM) is provided by the internal resonator which comprises the facets of the laser diode chip. These facets have relatively low reflectivity and therefore their transmission bands are relatively wide. The grating profile (GP) is defined by the properties of the specific grating used and by the correct alignment of the grating relative to the laser diode. Finally the external mode (EM) originates from the external cavity formed by the grating and the rear facet of the laser diode. Since the length of the cavity is much longer than that of the diode chip these modes are more closely spaced together). The emitted frequency (EF) of the external cavity laser diode system (100) will be at the point where the overlap of all gain profiles and modes give highest value, as illustrated in FIG. 7. Thus, the bandwidth of an external cavity laser diode system (100) such as the DL100, based on a NLHV500 laser diode, is about 4 MHz, in contrast to the bandwidth of 400 MHz obtained with the original free running NLHV500 violet laser diode (101).

An external cavity laser diode system as the DL 100 laser, particularly based on the NLHV500 diode, can operate at various radiation modes according to temperature and current conditions. In general four discrete operation states can be defined: (A) stable single longitudinal mode operation; (B) two stable longitudinal modes; (C) several longitudinal modes competing with each other; and (D) broad band operation. When considering the laser noise and the speckle visibility, which are the most important parameters for LDF measurements, the most appropriate operational state is state (A), with state (B) being less preferable though nevertheless possible. An intermediate state similar to state (B) can also exist, wherein 3 stable non-competing longitudinal modes are generated is less desirable. At operational states (C) and (D) the laser RIN noise is high, and therefore these operational states must be avoided during the laser Doppler flowmetry measurements.

In order to ensure that an external cavity laser diode system such as DL100 only operates in operation state (A) a special stabilization system is needed. This stabilization system implemented in the laser stabilization control system (LSC) (7), which is described in detail hereinbelow. This LSC system is an essential feature for the smooth and long-term continuous operation of the present invention, since the ECDL system (100) without the stabilization provided by the LSC system (7) will gradually drift between various states (A) through (D), as described above, and the laser Doppler flowmetry measurements obtained therefrom will thus be correspondingly unstable and unreliable.

Thus, in preferred embodiments of the present invention, and referring to FIG. 4, the apparatus, generally designated by the numeral (99), comprises a probe (2) operatively connected to a suitable laser source unit (LSU) (1), a detection unit—(DTU) (3), a signal processing and conditioning electronics unit—(EU) (4), a suitable computer (PC) (5) with suitable software, a suitable power supply—(PS) (6), and a laser stabilisation control system (LSC) (7). As will be described in greater detail hereinbelow, the laser source unit (LSU) (1) provides the correct tissue illumination—excitation conditions. The fiber optic probe (2) transmits the excitation light towards the tissue that is being examined or monitored, collects the reflection and fluorescence from the tissue and transmits it back towards the apparatus. The DTU (3) comprises appropriate optical filters and detectors for converting the collected light intensities to electronic signals from which typically up to four tissue vitality parameters may be monitored. The signals from the DTU (3) are fed into the EU (4) for processing. The EU (4) serves as a conditioning and signal processing system. It also converts the analogue signals to digital data that feeds into computer (5). The acquired data is processed by suitable software and may displayed by any suitable means and form, such as for example on the computer screen as charts and in digital form. The PS unit (6) provides each of the components of the apparatus (99) with the required electrical power. The laser stabilization controller (LSC) (7) controls the temperature and current of the laser diode in order to ensure that the external cavity laser system will work in stable single longitudinal mode (state (A)), though if necessary or desired, the LSC (7) may be configured to enable operation in state (B).

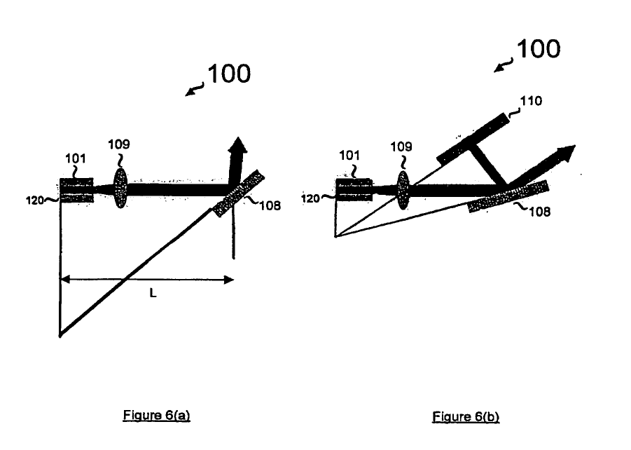

Referring to FIGS. 4, 8(a) and 8(b), the probe (2) comprises at the distal tip thereof a contact face (12) for making contact with the surface of the tissue (25) being monitored. In its simplest form, the probe (2) has a single fiber (201) for directing a laser radiation at a target excitation wavelength to nominally a point (15) on the tissue (25). Alternatively, a bundle of fibers may replace the single fiber (201). The laser radiation comes from a suitable source, such as violet laser diode (101) coupled to the fiber (201) by any suitable optical coupler that preferably enables selective coupling and decoupling of the probe (2) with respect to the rest of the apparatus (99), such as SMA connector (205). Preferably, the probe (2) comprises a plastic flexible housing in the form of tube (208) to protect the optical fibers, which are advantageously encapsulated within a stainless steel tube (209) at its distal tip.

The probe further comprises one, and preferably a plurality of, collection fibers (202) for collecting light from the tissue. When more than one detection fiber (202) is used, this plurality of fibers may be arranged on a circle, and in each case the fibers (202) are distanced (R1) from the excitation fiber (201) (or coaxially with the geometric center of the corresponding bundle of excitation fibers, where appropriate), as illustrated in FIG. 8(a). The number of collection fibers (202) as well as the core diameters of the fibers, influence both the average sample depth (SD) and the collected signal intensity, as well as the signal to noise ratio (S/N) of the laser Doppler measurements. For example, a 200 micron core excitation fiber (201) may be used together with four collection fibers (202), each of 100 micron core diameter and radial displacement (R1) of 0.20 mm from the excitation fiber (201). Collection fibers (202), are bundled together at the proximal end (266) and are provided with a common optical connector (267), such as an SMA connector, to enable convenient coupling/decoupling of the fibers with respect to the DTU (3).

The probe (2) is preferably disposable, but may be semi-disposable or non-disposable. The term “disposable” in the present application means that the probes are designed (in corresponding embodiments) to be disconnected from the rest of the apparatus (99) and thrown away or otherwise disposed off after one use with only negligible economic loss. Negligible economic loss herein means an economic loss per probe which is substantially less than that of the apparatus (99) itself, or of the medical costs associate with a procedure using said apparatus (99), or indeed of the costs associated with sterilising and reconditioning the probe for a single subsequent use. The term “semi-disposable” herein means that while the probe is disposable, it may nevertheless be used a limited number of times, with appropriate sterilising and reconditioning thereof between uses. The term “non-disposable” herein means that the probe is designed for multiple use, and is only disposed of when sterilisation and reconditioning thereof is no longer possible or economic. Thus, the probe (2) is typically designed for once-only use for minimising risk of cross-infection, for example. Optionally, though, the probe (2) may be adapted for sterilisation using an ETO or any other suitable sterilization technique, enabling the probe to be semi-disposable or non-disposable. In any case, the probe (2) is also typically made from biocompatible materials.

Radiation at nominally one wavelength, as will be further described hereinbelow, is delivered from the LSU (1) to the tissue (25) to be monitored via a single optical or excitation fiber (201) (or bundle thereof). The excitation fiber (201) and the collecting fibers (202) are placed in direct contact with tissue (25) in order to maximize the portion of light signal that penetrates the tissue and is subsequently collected from the tissue.

In one preferred embodiment of the present invention, the apparatus (99) is directed to the monitoring of blood flow rate, and at least one of and preferably all of NADH concentration, blood oxygenation state and blood volume pertaining to an identical tissue volume, and is described in detail hereinbelow. In another preferred embodiment of the present invention, the apparatus (99) is directed to the monitoring of blood flow rate, and at least one of and preferably all of Fp concentration, blood oxygenation state and blood volume pertaining to an identical tissue volume.

Thus, in the first preferred embodiment of the present invention, the photons of the penetrating light undergo scattering and absorption as they interact with the body tissue matter. The scattering of excitation light is mainly due to interaction with stationary tissue and with the red blood cells. The absorption of the excitation light is mainly due to tissue and blood haemoglobin, and to a lesser extent is due to NADH molecules. Some of the energy that is absorbed by NADH is re-emitted by NADH molecules as fluorescence photons, a small portion of whom eventually reaches the tissue surface, and are collected by one or more collection fibers (202) and transmitted to the DTU (3). Doppler shift changes in the radiation give a measure of the blood flow rate, and such changes are detected via one or more said collection fibers (202).

Referring to FIG. 4 and FIG. 6(a), the LSU (1) according to the first preferred embodiment of the present invention comprises a suitable violet laser head (100) preferably capable of generating a single longitudinal mode narrow bandwidth laser radiation at about 390 nm. The laser head (100) comprises a stabilized single longitudinal mode laser diode or an external cavity stabilized laser diode system (ECLD) laser head (100), such as DL100 from Toptica Photonics AG, Munich, Germany, for example. The ECDL system laser head (100) comprises a suitable violet laser diode (101), such as NLHV500 from Nichia Chemical Industries Ltd., Anan, Japan. A collimating lens (109) collimates the laser diode light toward the grating (108). The grating (109) serves as the front mirror of the ECLD system resonator while the rear facet (120) of the laser diode (101) comprises the back mirror, providing an increased resonator length (L). The grating settings for the grating (109) illustrated in FIG. 6(a) may be according to the Littrow Design, or alternatively, a different configuration for the grating (109) may be used, in conjunction with a tuning mirror (110), according to the Metcalf-Littman Design, mutatis mutandis, as illustrated in FIG. 6(b). A small portion of laser radiation passes through the rear mirror (120) and reaches the intensity monitoring photodiode (102). The photodiode (102) is a part of the laser diode, and preferably it is intergral with the laser diode (101). The ECLD system (100) preferably further comprises also a Peltier cooler and temperature stabilization controlling circuit (not shown) that is operated by the temperature controller (701). In the preferred embodiment, the ECLD system (100) operates in continuous wave (CW) mode.

The laser radiation from the laser head (100) is reflected by a suitable mirror (111) towards an acousto-optic (AO) modulator (103). In the preferred embodiment, the AO modulator (103) enables fast chopping/modulation of the laser light, as will be described in more detail hereinbelow. The CW laser radiation is chopped by the AO modulator at a repetition rate of 4 KHz to provide a stream of substantially identical pulses of laser light. In the preferred embodiment, each cycle duration is 250 microsec with a duty cycle of 1:10, i.e., with the ON period duration being 25 microsec, and the OFF period being 225 microsec. The chopping sequence is generated by the clock (403), which is comprised in the EU (4). The chopped radiation appears at the 1st order of the (AO) modulator (103). This order is spatially filtered by a circular diaphragm (not shown). The modulated radiation from the AO modulator (103) is reflected by dielectric mirror (106) and coupled to excitation fiber (201) of probe (2) by the lens (104) mounted on a suitable coupler adapter (105). A small portion (typically about 1%) of the excitation radiation passes through the dialectric mirror (106) towards the photodiode (107). This photodiode (107) enables the excitation intensity that reaches the probe (2) to be monitored. For enhanced safety a mechanical safety shutter (113) may be provided for preventing the laser radiation from reaching the probe (2) when the apparatus (99) is not in measurement mode or when the probe connector (205) is disconnected from the coupler (105). Preferably, operation of the safety shutter may be controlled by means of computer (5) via A/D (401).

The laser stabilization controller (LSC) (7) controls the temperature and current of the laser diode (101) in order to ensure that whole ECDL system (100) will operate in stable single longitudinal mode (state (A)), though if necessary or desired, the LSC (7) may be configured to enable operation in state (B). Thus, the LSC (7) unit comprises all sub-components needed for operation of the laser diode (101), and especially the laser head (100), which typically comprises a ECDL system, as described herein. The LSC (7) comprises a temperature controller (701) that includes the feedback of temperature data so as to enable the target to be heated or cooled, as appropriate to maintain a nominal temperature, and a highly stable current controller (702) for the diode laser (101). Additionally the LSC (7) further comprises a micro-controller, typically a microprocessor (704), with Analog to Digital (A/D) (703) and Digital to Analog (D/A) (705) converters. The A/D converter (703) receives the laser intensity proportional voltage from the photodiode (102). This analog voltage is converted to digital form and is fed into the microprocessor (704) as digital data. After processing the laser intensity information according to a predefined algorithm, the microprocessor (704) determines the precise value for the laser current and temperature that is required, and provides corresponding analogue signals, through the D/A converter (705), to the current controller (702), which in turn powers the diode (101). Various algorithms may be devised for finding and maintaining the operational state (A) for the ECDL system laser head (100). Perhaps the simplest such algorithm may be based on the measurement of the RIN parameter of the ECLD system laser head (100). As discussed above, the RIN is in essence the ratio between the (AC) fluctuations of the laser intensity to the total laser intensity (DC). Therefore, the RIN parameter is easily available from the measurement of the AC and DC components of the output voltage of the photo-detector (102). In order to maintain stable operational state (A), the microprocessor (704) controller initialises a short sweep of the laser current dI around the predefined nominal current I0. For example the NLHV500 operates at a nominal current I0=40 mA, and the current sweep dl may be about ±1 mA, for example. During this current sweep the ECDL system of the laser head (100) passes through all Operating States (A) through (D). Since the desired state (A) has the smallest RIN the current regimes associated with each state may be easily determined. After determination of the current regime associated with state (A), the center of the regime can be calculated and the laser current to laser diode (101) can be changed accordingly.

The ECLD system stabilization procedure can be initialized immediately at system power ON and with every calibration procedure of measured parameters as will be described hereinafter. In general after ECDL system is forced to operate at a stabilized state, it will in general remain there for several tens of minutes. During this period the system should preferably be re-stabilized periodically in order to force it to remain in such stabilized state in the long term. This re-stabilization can be performed during the OFF periods of the laser pulses in state II or III as will be described later. The duration of the ECLD system stabilization procedure is less then 0.4 sec therefore it can be easily performed in between the trains of pulses of state II or III.

The light from collecting fibers (202) is coupled to the DTU (3) via optical connector (267). The light from the connector (267) is collimated by lens (306) within the DTU (3).

The light collected by the collection fibers (202) consists mainly of reflected light at the excitation wavelength, but it also comprises much lower intensity NADH fluorescence light at higher wavelengths. The portion of the collimated beam comprising the reflected light will thus have the lowest wavelength, corresponding to the excitation wavelength, while at the same time having the highest intensity of the radiation collected by the collecting fiber (202). Thus, the first dichroic mirror (302) splits off light at the excitation light wavelength from the collimated beam, channeling this portion of the beam towards a low-noise, fast photodiode detector (301), such as a Hamamatsu S5973-02 detector. Preferably, a condensing lens (305) is used in order to fill the photo-detector active area. The dichroic beam splitter (302), therefore reflects most of the light at excitation wavelength and while permitting transmission therethrough for most of the higher wavelengths in the collimated beam, and thus provides enough filtration for the photodiode detector (301), with no additional filtration being generally needed. The signal from the photodiode detector (301) is used to perform reflection measurements to determine the blood volume tissue viability parameter, and to perform Doppler flowmetry measurements to determine the corresponding blood flow rate. The remainder of the collimated light beam continues towards the second dichroic mirror (303).

Thus, radiation of wavelengths higher than the excitation wavelength passes through the dichroic mirror or beam splitter (302) and is incident on a second dichroic beam-splitter (303), which is selected to reflect wavelengths lower then about 440 nm and to transmit all higher wavelengths. The reflected light beam is passed through a suitable filter (307), preferably a 435 nm (10DF) filter, and is then fed into a first photo-multiplying tube (PMT) (308). The light transmitted through the second dichroic beam-splitter (303) is subjected to additional splitting by a third dichroic beam-splitter (304) that reflects wavelengths lower than 460 nm, but is transparent to higher wavelengths. The reflected light from the third dichroic beam splitter (304) is filtered by a suitable filter (309), preferably a 455 nm (10DF) filter, and is then incident on a second photo-multiplying tube (PMT) (310). This wavelength is close to an oxy-deoxy isosbestic point, so the fluorescence intensity as measured by this PMT (310) correlates directly with the NADH fluorescence. The light that passes through the third dichroic beam-splitter (304) is subsequently filtered by a suitable filter (311), preferably a 475 nm interference filter (DF10), and the filtered light is incident on a third photo-multiplying tube (PMT) (312). The precision of all above-mentioned filters are ±5 nm.

The fluorescence intensity measurements provided by the first, second and third PMTs (308), (310) and (312) respectively, are used to determine the blood oxygenation state, i.e., the ratio of oxygenated blood to deoxygenated blood, within the tissue element, according to the method described in co-pending Israel Patent Application No. 138683 filed by Applicants, the contents of which application are incorporated herein in their entirety, mutatis mutandis.

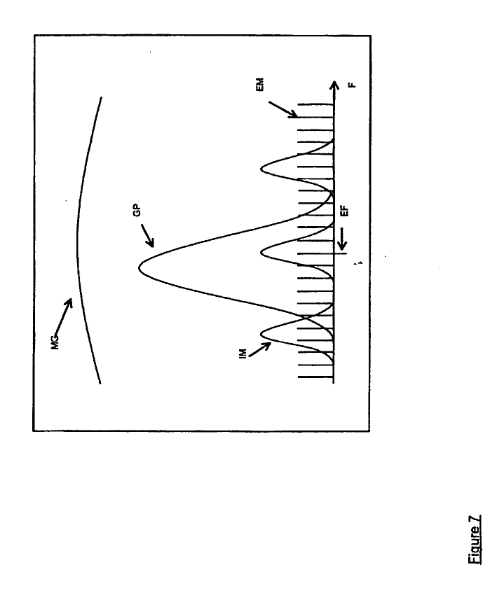

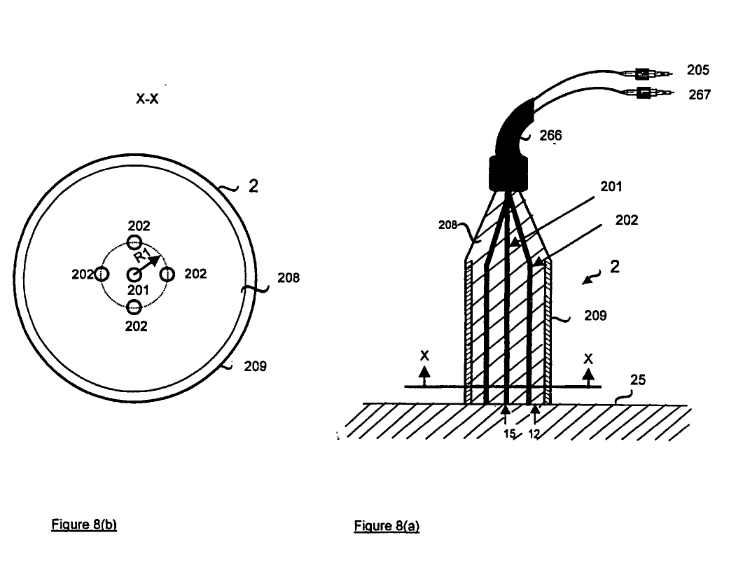

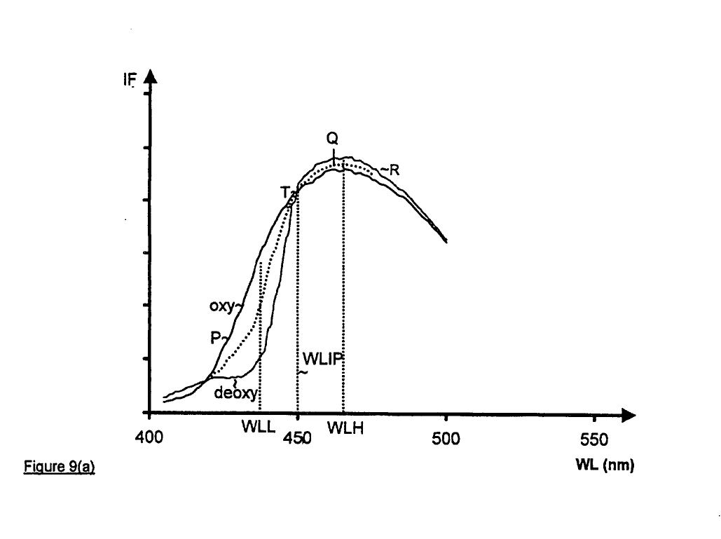

Thus, as far as blood oxygenation measurements are concerned, any suitable illumination wavelength may be used, and fluorescence that is emitted from the tissue element is then monitored. When combining the blood oxygenation measurements with NADH measurements, and particularly with blood volume measurements, the wavelength of the illumination radiation is advantageously chosen to correspond to a suitable isosbestic point. The intensity of the fluorescence emitted, as a function of wavelength, will vary according to the blood oxygenation state of the tissue element. Thus, referring to FIG. 9(a), curve (P) represents the intensity of the fluorescence (IF), emitted for the full range of wavelengths (WL) of the emission when the blood in the tissue element is fully oxygenated, while curve (R) shows the corresponding (IF)-(WL) relationship for the fully deoxygenated condition. Curve (O) represents an intermediate condition in which the blood is partially oxygenated and partially deoxygenated. All the curves pass through point (T), which is herein referred to as an “oxy-deoxy” fluorescence emission isosbestic point, corresponding to a fluorescence wavelength of (WLIP). This oxy-deoxy fluorescence isosbestic point will be at the same wavelength as the isosbestic point of oxy-deoxy haemoglobin absorption spectrum namely about 455 mm±5 nm as shown in FIG. 3. By measuring the ratio of the intensity of the fluorescence at a wavelength below (WLIP), say at (WLL) to the fluorescence intensity at (WLIP) and also at a higher wavelength, the ratio of the intensity at say (WLH) to the intensity at (WLIP), the actual blood oxygenation state can be determined. Thus, and referring to FIG. 9(b), if the fluorescence intensity ratio IF(WLL)/IF(WLIP) is increased, then the blood is substantially mostly oxygenated, while the converse is true if the intensity ratio decreases. An increase in the fluorescence intensity ratio IF(WLH)/IF(WLIP), indicates that the blood has become more deoxidized. By suitable calibration, particularly of the maximum and minimum intensities (IF) at these wavelengths, the actual relative percentages of oxygenated to deoxygenated blood may be determined given the fluorescence intensities measured at these points. In order to maximize the sensitivity and precision of the method the (WLL) and (WLH) should be chosen in such a way that the change of the ratio IF(WLL)/IF(WLIP) and IF(WLH)/IF(WLIP) will be maximised with respect to oxy-deoxy relative concentration variations. Therefore, the wavelengths where this change is maximal, (WLL)_{MAX} and (WLH)_{MAX}, should be used. Indeed, greater sensitivity to even minor changes in blood oxygenation may be achieved by monitoring the ratio of the aforementioned ratios IF(WLL)/IF(WLIP):IF(WLH)/IF(WLIP) which is, of course, equivalent to the ratio of IF(WLL)/IF(WLH).

Thus, blood oxygenation level is provided by the first, second and third PMTs, (308), (310) and (312) respectively, wherein the second PMT (310), in which fluorescence intensity is measured at an isosbestic point, also provides the NADH parameter. Thus, the ratio of the fluorescence intensity measured by the first PMT (308) to the intensity measured by second PMT (310), generally increases as the blood becomes more oxygenated, while the ratio of the fluorescence intensity as measured by the third PMT (312) to the intensity measured by the second PMT (310) under the same conditions will decrease. Conversely, as the blood becomes more de-oxygenated, the fluorescence intensity ratios measured by the first PMT (308) and by the third PMT (312) relatively to the intensity measured by second PMT (310) generally will decrease and increase, respectively. The measured fluorescence ratios can be calibrated to actual levels of oxy-deoxy haemoglobin using measurements by other known methods, such as pals-oximetery. Thus relative levels of oxygenated blood to deoxygenated blood within the tissue element may be determined.

The electronic and electro-optic components described herein are given by way of example. There are many alternative methods of realizing the current invention. For example, although the monitoring of the three parameters NADH, blood volume and blood oxygenation state, is accomplished with PMT detectors, optical filters and dichroic splitters in the embodiment described herein, it is possible to replace all these components by using a grating spectrometer and appropriate detector such as a CCD or by using a multianode PMT with a Multi-band interference filter such as Hamamatsu R5900F-L16. These solutions could potentially monitor intensity ratios with even higher precision, but at current prices, are not economical options.

By way of example, a suitable component for the PMT detector modules (308), (310) and (312) is the Hamamatsu 6780 PMT. Each of the PMT detector modules (308), (310), and (312) comprises a PMT tube and all electronics necessary for the PMT gain control. These modules are supplied with the operation voltage and each module has gain control input and signal output connections. The electronics circuits for all 3 PMT detectors are identical.

The signal output of the PMT detector (312) is fed to the signal conditioner (402) input. There are several ways of accomplishing the signals processing which are well known in the art. All detectors in the proposed system are synchronous detectors. The appropriate electronic circuit is described below.

The monitoring of all parameters, LDF, NADH fluorescence, blood volume and blood oxygenation level involve excitation light at about 390 nm, which is in the UVA spectral region. Exposure to UVA radiation should be minimized as it is considered to be potentially dangerous even at low irradiation levels. In order to reduce the radiation safety problem, the option is provided in the present invention to chop the excitation light with a duty cycle of 1/10 (ON Time/Total Cycle). Additionally use of chopped light enables the employment of synchronous detection techniques that enable better signal detection and recovery from noise.

The bandwidth of the laser Doppler flowmetry signals is from several tenths Hz to several KHz. This bandwidth imposes the lowest suitable chopping frequency according to the Nyquist principle. In practice the present inventors found that a 4 KHz-chopping rate is sufficient for typical laser Doppler flowmetry measurements.

Still after applying the chopping technique in order to reduce the tissue irradiation during long procedures or during the use of the apparatus in Intensive Care Unit (ICU), the total amount of irradiation applied to the tissue can be even more reduced by using the adaptive chopping technique.

There are many clinical conditions such as at long procedures or during ICU hospitalization where true real-time on-going measurements are unnecessary. At these stable state periods the measurements can be performed for only second set of parameters namely NADH fluorescence, blood volume and blood oxygenation level, since for measurement of these parameters only few sampling events are needed. The measurements of LDF must be performed by many sampling events at high repetition rate, since it requires a measurement of signals of a bandwidth of at least several KHz. This relatively demanding measurement of LDF can be omitted during the steady state period.

Thus, whereas during critical parts of a surgical operation procedure the output data should be renewed at least at the rate of two data points per second, there are however, many cases where the patient's condition is stable, so that a data sampling rate of only, say, once every two seconds is required.

Thus, in order to reduce the tissue irradiation the apparatus (99) according to the present invention may be operated in any one of several irradiation modes, and corresponding to these modes are several data acquisition modes. There are two basic concepts behind these operation modes:

The first concept relates to monitoring that is perceived to be continuous by the clinical personnel. In general, all vitality signals data should be presented to the medical personnel in real-time. That is, the device display should be updated at the rate that reflects the real physiology events as they evolve in the patient. This means that if for example the patient is in a critical stage of the surgery and there are a lot of fast changes in the physiological conditions, the screen update rate should be fast i.e. about two data points per second. However, where the patient is in a more stable condition such as at the beginning of the surgery, at its final stage or in the intensive care unit (ICU), the vital parameters will generally tend not to change very fast, and therefore a much slower screen update rate can be utilized. In such cases the update rate can be for example one data point every 2 seconds.

The second concept is that actually all vital parameters are mutually connected and inter-related. Therefore a change in one parameter should immediately trigger a change in at least one other parameter. Especially any change in the blood flow will be accompanied by a change in at least one of the other parameters: blood volume, blood oxygenation or the NADH fluorescence. This means that if the patient's state is steady, such as in ICU, the monitoring of the blood flow can be stopped for long periods whilst all the other tissue vitality parameters are monitored. Where any significant change in the value of any one of these parameters is detected the system will automatically start monitoring of all parameters including the blood flow, until a steady state is again reached.

Thus, according to the present invention the apparatus (99) may be used according to an adaptive chopping procedure. In such an adaptive chopping procedure, the radiation provided source (101) may be chopped to provide corresponding pulses of radiation at the appropriate wavelengths, the pulses being provided at a preferably variable frequency of pulsation, i.e., chopping frequency. It is important that this form of “pulsing” is different from the “pulsed” laser mode commonly encountered. The “pulsing according to the present invention provides a great degree of uniformity between the pulses generated as a result of the chopping procedure. Regular pulsed lasers cannot generally provide such a high level of uniformity between pulses generated thereby. Furthermore, the apparatus (99) may be further adapted such that packages of pulses may be provided as and when required or desired. Such packages may each comprise a variable number of pulses, and the time interval between packages of pulses may also be independently controlled. Thus, at periods where relatively little monitoring is required, few packages containing a few pulses each may be transmitted with large “OFF” intervals in-between packages (i.e., where no radiation is provided), while at other, more intense periods, the packages of pulses may be sent with little or no intervals between successive packages. By pulsing, and by also packaging the pulses as described, the radiations provided by source (101) may be of a higher permitted intensity than would normally be allowable, albeit for shorter duration. This results in better signal-to-noise ratios of the signal, as well as to safer radiation levels for both the patient and the operators of the apparatus and equipment.

As described in greater detail hereinbelow, using the concept of adaptive chopping, it is possible to entirely stop the laser Doppler measurements after this parameter has reached a steady state. The remaining three parameters, the second group, may be measured by providing short packages of pulses at a frequency of, say, once a second, which while sufficient for monitoring NADH concentration, blood oxygenation state and blood volume, are too low for blood flow measurements, and thus minimise exposure to the laser radiation. Indeed the second set parameters—NADH concentration, blood oxygenation state and blood volume—will also be in steady state until some change occurs. If the change originates in the blood flow rate, it will immediately induce a change in the other, actively monitored parameters, such as the blood volume. The apparatus (99) may then be configured such that when such a change is detected, the Laser Doppler measurements automatically restart and continue until at least the next steady-state condition is reached.

The outputs of the photodiode detector (301) and the outputs of the three PMT detectors (308), (310), (312) are connected to the signal conditioner (402). The signal conditioner (402) receives synchronization signals that correspond to the chopping sequence from the clock (403). The signal conditioner features three groups of ‘channels’ or synchronous detector circuits, which will be described below.

The signal conditioner (402) of the EU (4) converts the chopped signals into continuous wave (CW) signals. These are converted by the A/D unit (401) into digital data, which is then fed into the computer (5) through the analog input output (AIO) ports. The A/D sub-unit (401), besides digitizing the analog measured signals, also enables the receiving of digital commands from the computer (5) via the digital input output (DIO) ports.

The clock (403) sub-unit provides the appropriate timing for the AOM (103) and the signal conditioner (402).

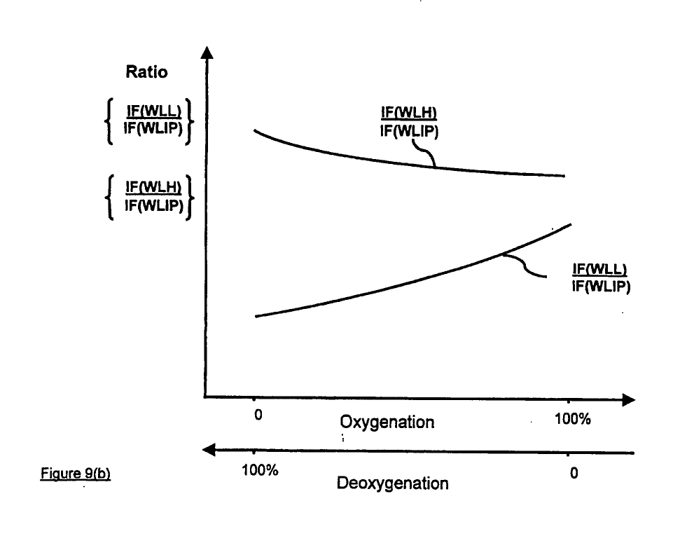

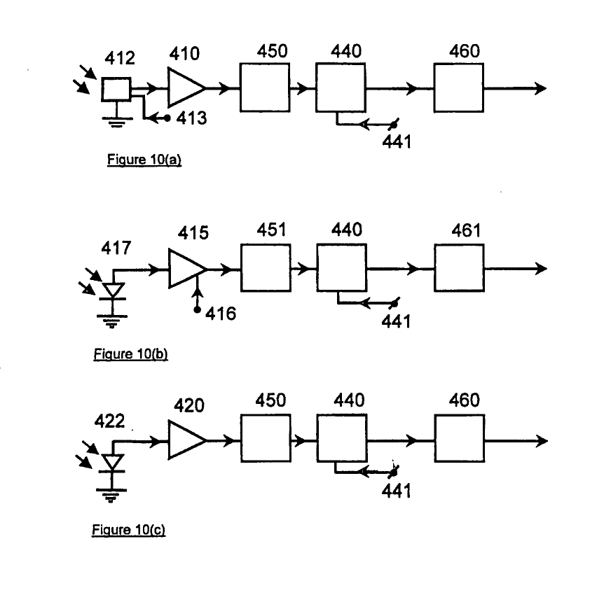

Referring now to FIGS. 10(a), 10(b) and 10(c), In a specific, non-limiting example of the preferred embodiments brought for illustrative purposes, three types of optical detectors with corresponding electronics circuits are used.

The first type of detector as shown in FIG. 10(a), is a photon multiplier tube (PMT) detector (412). This detector type is suitable for use as components (308), (310), (312) shown in FIG. 4. These detectors are used for NADH fluorescence measurements. The detector may be built around a PMT module from Hamamatsu H6780. This integrated module consists of PMT tube, a high voltage power supply and all necessary control electronics. One need only to supply the operating voltage and the control voltage for the gain control, and the module itself changes the high voltage of the PMT accordingly. As illustrated in FIGS. 4 and 10(a), the gain of such detector (412) may be controlled by gain input (413) by the PC (5) through the A/D (401) unit. The output of this PMT module is fed to the inverter (410), since the module produces negative output relative to ground. The output of the inverter is fed to narrow band pass filter (450). The central frequency of this filter is 4 KHz. The purpose of this filter is to avoid aliasing of detector noise at higher frequencies unto the detector band pass and to avoid detection of various noise signals at frequencies different from the chopping frequency. After filtration the signal is sampled by a sample and hold (S/H) circuit (440) built around S/H such as Analog Devices AD781. The sample event is initialized by the clock (403) (FIG. 4) via the (441) input. The sampled signal is fed to the Low-Pass filter (460). That Low-Pass filter enables averaging in time of the measured light intensity, and thus it reduces the signal fluctuations. The output voltage of the Low-Pass filter (460) is proportional to the light intensity impinging the PMT detector.

The second type of detector, illustrated schematically in FIG. 10(b), is a fast photodiode detector such as (301) (see FIG. 4). This kind of detector is used for reflection and Doppler measurements. This type of detector is built around Hamamatsu S5973-02 photodiode (417) (see FIG. 10(b)) connected to a pre-amplifier (415). The pre-amplifier (415) consist of trans-impedance amplifier such as Analog Device AD743 and additional amplifier with controllable gain such as Motorola MS30340. The gain of this detector type is controlled via gain input (416) The output of the pre-amplifier (415) is fed to Band Pass filter (451). This filter (451) is a Band-Pass filter. The central frequency is 4 KHz while the bandwidth is 3 KHz. The purpose of this filter is to avoid aliasing of a detector noise at higher frequencies into the detector band pass and to avoid detection of various noise signals at frequencies different then the chopping frequency. The bandwidth of this filter chosen to enable detection of Doppler signal at frequencies from several Hz to 1.5 KHz. The output of the Band-Pass filter (451) is fed to S/H circuit build around S/H such as Analog Devices AD781 (440). The sample event is initialized by the clock (403) (FIG. 4) via the (441) input. The sampled signal is fed to the 1.5 KHz Low-Pass filter (461). The DC level of the output of the Low-Pass filter (461) is linearly proportional to the light intensity at the detector, thus it represents the reflection value. The AC ripple over imposed on this DC level. The Doppler processor at EU (4) analyzes the AC component along with the DC level and computes the LDF value according to the well known algorithm disclosed elsewhere (Bonner R. F. and R. Nossal. Principles of laser-Doppler flowmetry. In: Laser-Doppler blood flowmetry, edited by A. P. Shepherd and P. A. Oberg, Boston, Dordrecht, London:Kluwer academic, 1990, p. 17-45).

The third type of detector, illustrated in FIG. 10(c), is a fast photodiode detector. This is suitable for use for photodiode detector such as (107) at FIG. 4. This type of detector is used for light source intensity measurements. The light source intensity information is used at the final stage of data processing to normalize the reflection and fluorescence intensities according to the changes in the light source intensities. This type of detector is built around a Hamamatsu S5973 photodiode (422) (see FIG. 10(c)) connected to trans-impedance amplifier such as Analog Devices AD713 (420). The output of the trans-impedance amplifier (420) is fed to a Band-Pass filter (450). The central frequency of this filter is 4 KHz. The purpose of this filter is to avoid aliasing of a detector noise at higher frequencies into the detector band pass and to avoid detection of various noise signals at frequencies different then the chopping frequency. The output of the filter (450) is fed into the sample and hold (S/H) circuit build around S/H such as Analog Devices AD781 (440). The sample event is initialized by the clock (403) (FIG. 4) via the (441) input. The sampled signal is fed to the Low-Pass filter (460). That Low-Pass filter enable averaging in time of the measured light intensity, thus it reduces the signal fluctuations. The output voltage of the Low-Pass filter (460) is proportional to the light intensity of the laser source. Since the range of the intensities involved for the light source is known ahead the gain of this kind of detector is set constant to appropriate value.

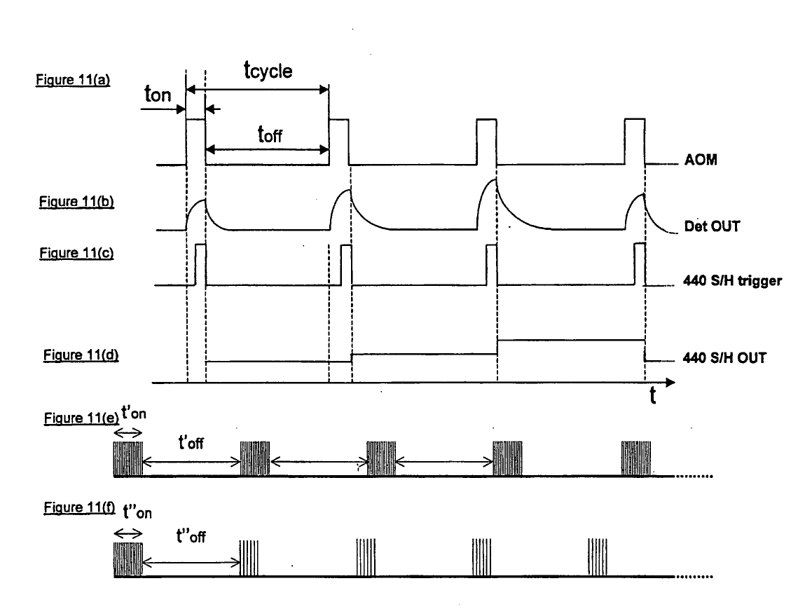

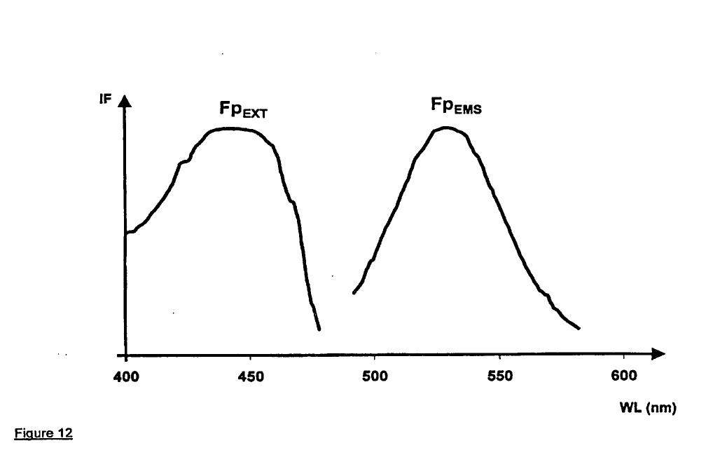

Referring to FIGS. 11(a) to 11 (d), the trigger signal timing in FIG. 11(c) provided by the clock (403) is correlated with the end of the light ON period in FIG. 11(a) when the output voltage of FIG. 11(b) of the detector is at a maximum, enabling the S/H circuit to sample the maximum available signal. The S/H circuit (440) holds this voltage value of FIG. 11(d) until a new trigger signal shown in FIG. 11(c) arrives from the clock.

The gain of the detectors is defined automatically by the accompanying software in computer PC (5), according to the detected light intensity values. If the detected light signal is too small, the software provides an appropriate signal to increase the detector gain as described below. There is a difference in the gain management of the three types of the detectors as described above. The gain of the first detector type, the PMT, is set by changing the control voltage (413) of the PMT module (412). This actually changes the sensitivity of the PMT detector. The setting of the control voltage is performed by the software that runs on the PC (5) through the analog to digital converter (A/D) module (401) of the electronics unit (EU) (4). This A/D and D/A module can be any one of the variety of cards produced by National Instruments and other manufacturers.

The gain of the second detector type is set by changing the gain of the second stage of the pre-amplifier (415) gain rather then by changing the sensitivity of the photodiode detector itself. The setting of the control voltage is performed by the software that is adapted to run on the PC (5) through the A/D module (401) of the electronics unit (EU) (4).

The gain of the third detector type is constant since this detector measures light source intensity having a predefined value that suits the constant dynamic range of the detector.

The gain setting procedure is initiated by the calibration command from within the device software. The calibration signal arrives from the computer (5) via digital to analog converter D/A (401). At the beginning of the calibration procedure the gain control voltage of the first and second detector type is reduced to zero, and then, the gain gradually begins to increase whilst the intensity of the output signal is monitored. With reference to the output of the detectors (308), (310), (312) and the detector (301) in FIG. 4 each detector gain is set separately. When the output voltage reaches about 2V, the gain is locked to the current value. This gain value is monitored by the software through the analog to digital converter A/D (401). From then onwards, any change in collected light intensity is monitored by the circuit and is transformed to digital information by (A/D) (401). Since the gain value, is known, the actual light intensity may be calculated and displayed on the screen by the software.

The clock sub-unit (403) typically comprises a programmable clock. According to computer input via bus (404) the clock output will be in one of the following states (with particular reference to FIG. 11(a)-11(f)):

State I: The clock signal consists of a train of pulses in FIG. 11(a). The ON period tₒₙ of the cycle is 25 microsec, while the whole cycle t_{cycle} is 250 microsec i.e. the repetition rate is 4 KHz. Therefore the duty cycle is 0.1. This sequence shown in FIG. 11(a) is used for enabling the light source (102) by the triggering of the AO modulator (103), and also is used, after appropriate delays, to trigger the signal S/H circuit (440) by sequences in FIG. 11(c). The sequence in FIG. 11(c) is correlated to the end of each pulse shown in FIGS. 11(a). Thus the sequence in FIG. 11(c) enables sampling by all detectors (107), (301), (308), (310) and (312) (see FIG. 11(a)-11(f) and FIG. 4). FIG. 11(b) shows a typical detector response to an excitation light pulse.

State II: State I is additionally chopped by an ON/OFF adaptive duty cycle which enables and disables the light pulses train of FIG. 11(a) as shown in FIG. 11(e). During the ON period t′ₒₙ (0.1 sec) of the adaptive duty cycle, 400 pulses (FIG. 11(a)) of 10 microsec each are generated. The OFF period t′_{off} of the adaptive duty cycle is controlled by the computer. The OFF period can be 0.4 sec for relatively fast-changing conditions and can be prolonged to as much as 5 sec for slow changing conditions. The t′_{off} is determined automatically by the software to minimize the total tissue irradiation.

State III: The clock generates a sequence of five cycles of the state I like the pulses shown in FIG. 11(a). These ten pulses are used for enabling light source (101) by the AOM (103) and all the detectors (107), (301), (308), (310) and (312) on FIG. 4. At this state of the clock, measurements of only the second set of parameters are enabled, i.e., of NADH, blood volume and blood oxygenation state. Doppler flowmetry measurements are not performed since there are too few data points available therefor. This State is particularly useful for very long steady state measurements, such as in an Intensive Care Unit.

The device software controls the tissue sampling and irradiation. At measurement initialization the clock is in state I, enabling the correct setting of the gain for all detectors, and the normalization of the output signals. After a short time, if fast changes in any one or more parameters are observed the clock is switched to state II, having a short OFF period t′_{off}. After the changes became more moderate, the OFF period t′_{off} becomes longer. After cessation of the changes as steady state is achieved, the system switches to state III in order to minimize the tissue irradiation. Detection of changes causes the system to switch back to state II.

Of course, the apparatus (99) can be configured to operate only in State I, either permanently, or whenever desired, without resolving the tissue irradiation safety issues in particular regarding internal tissue sensitivity to the UVA radiation. The current laser safety standards define only standards for the skin and eyes, but information is still lacking regarding the limiting values for the irradiation of internal tissues.

The PS (6) typically comprises an on-line medical grade power supply with an insulating transformer as required by Standard IEC 601-1 for electrical medical equipment.

The PC (5) typically comprises a Pentium II or higher system running Windows 95/98/NT or higher. The dedicated Computer and Power Supply are specified to meet EMC and other requirements for medical apparatus.

The dedicated software for the PC (5) is preferably based on the National Instruments LabView platform. The Doppler module calculates the blood flow according to well-established algorithms. The Exposure Tracking module calculates the total and the mean exposure. It also decides in which of the three possible clock modes the system will operate. When stable signals are detected for all measured parameters, the system will switch to State III. In that mode the tissue receives extremely low exposure. Only three parameters are measured i.e. NADH fluorescence, Oxygenation and Reflection. The blood flow rate is not actively monitored. If a change is detected in the value for any one of the measured parameters, this module switches the system to State II where all four parameters are actively measured. When calibration is initiated the system is switched to State I where all four parameters are measured at high sampling rate.

The system or apparatus (99) may be operated as follows: At the beginning of the measurements the user places the probe (2) on the tissue (25) and activates the system via a terminal of the computer. This automatically initiates a calibration sequence that lasts about 1 sec. During the calibration sequence the gain of the detectors are established and fixed. During calibration sequence, the clock generates pulses according to state I.

At the end of the calibration, the computer switches the clock to state II.

When switched to state II the OFF period is set to 0.4 sec so that the system measures all parameters at the rate of 2 data points per second. If after 10 readings, (i.e. 5 sec) there is no substantial change in any of the parameters, the OFF period t′_{off} is gradually increased to a maximum of 5 sec. If a steady state is attained, the clock is switched to state III. In state III ten 25-microsecond pulses are generated according to state I. Although this low number of pulses is insufficient for laser-Doppler measurement, it is sufficient for Reflection, Fluorescence and Oxy-Deoxy measurements. The pulse packets of state III are initiated every 0.5 sec to 6 sec depending on the monitoring mode, or until a physiologically significant change, such as, say, a 2% change in the value of any of the three parameters monitored. This 2% change is measured relative to the value of the parameters as measured in the last state II event. After leaving state III, the system switches to state II with an OFF period of 0.4 sec.

Advantageously, an ECLD system stabilisation procedure can be initialized immediately at system power ON and with every calibration procedure of the measured parameters. In general after the ECDL system is forced to operate at a stabilized state, by means of the LSC (7), it will in general remain at this stabilised state for several tens of minutes. During this period the system should preferably be re-stabilised periodically in order to force it to remain in such stabilized state in the long term. This re-stabilization can be performed during the OFF periods of the laser pulses in state II or III, described above. The duration of the ECLD system stabilisation procedure is typically in the order of less then 0.4 sec, and therefore such stabilisation can be easily performed in between the trains or packages of pulses of state II or state III.

In routine clinical use the system is preferably used in states II and III, with the mean irradiation being typically less than 0.5 mW/cm².

Thus, tissue may be irradiated with chopped light to provide important advantages, such as improving the accuracy in the measurements for all four parameters that are being monitored. Chopping enables the peak illumination intensity to be increased while holding constant the average intensity of the excitation. It allows the average excitation intensity to be reduced to within safe limits with respect to photo-damage. This can be achieved without significant loss of reasonable signal to noise levels.

“Chopped” laser illuminating radiation may be produced by chopping the excitation light illumination, and this may be achieved, for example, by an Acoustic Optic Modulator (AOM), though a fast rotating chopper wheel or any other chopping device may also serve this purpose. Similarly, direct modulation of the light source current could be used to generate the chopping effect.

In the context of this specification the duty ratio (DR) of the pulsed excitation is defined as the ratio of the duration of each pulse to the total cycle time. When the duty ratio is decreased, the signal to noise ratio is increased by factor (DR)⁻¹ for a parameter whose measurement is limited by background noise and by factor of (DR)^{-1/2} for a parameter which signal quality is limited by white noise generated in detection apparatus (Hodby J., J. Physics E: Scientific Instruments, 3, 229-233, 1970).

The apparatus according to the present invention is based on a violet laser diode having moderate power consumption, and may be designed to occupy a reasonable volume such as to be easily and conveniently transportable and also storable within a regular operating theater, for example.

In the second preferred embodiment of the present invention, the apparatus (99) is adapted for monitoring blood flow rate and at least one of flavoprotein level, blood volume and blood oxygenation state based on the flavoprotein parameter, and thus comprises all the components and in the arrangement thereof similar to that of the first preferred embodiment as described above, mutatis mutandis, with the following differences.

Whereas in the first embodiment the illuminating wavelength is between about 370 nm and about 400 nm, and preferably 390 nm, so as to lie within the NADH excitation spectrum, in the second embodiment the illuminating wavelength is chosen to be between about 400 nm and about 470 nm, and preferably about 440 nm, so as to lie within the flavoprotein excitation spectrum, and preferably at an Fp isosbestic wavelength within the flavoprotein excitation spectrum at about 455 nm±5 nm.

Essentially, the Fp-based measurements are very similar to those based on the NADH parameter. The Fp fluorescence excitation is by monochromatic light at a wavelength within the Fp excitation spectrum, preferably at an Fp isosbestic wavelength thereof. In the present invention, this monochromatic light is provided by, and at the wavelength of, the laser light source. The Fp fluorescence is measured by measurement of fluorescence intensity of the fluorescence emission at single wavelength, which is within the emission fluorescence spectrum. This Fp fluorescence intensity provides a measure of the Fp concentration in a similar manner to that the derivation of NADH concentration from NADH fluorescence intensity, mutatis mutandis.

As with the NADH fluorescence parameter, problem of haemodynamic artifact is also relevant to Fp measurements, and compensation for this artifact is similar to that for the NADH measurements. For the Fp parameter, reflection is measured at the wavelength of the excitation of the Fp fluorescence. This wavelength, in the present invention, is also the wavelength of the Doppler LDF measurement. In the embodiments described herein, the same detector that measures Doppler LDF also measures the reflection at the same wavelength since it is the intrinsic Doppler measurement that consist of measurement of AC signal that is superimposed on the DC reflection signal. This reflection value is subtracted from the Fp fluorescence value (in the same manner as in NADH measurements) in order to get corrected Fp fluorescence values. This typifies the compensation procedure.

As with the NADH parameter, it is preferable to measure the Fp emission (fluorescence) at oxy-deoxy isosbestic points such as 530 nm or 546 nm. Otherwise the fluorescence value will be influenced by the blood oxygenation. Similarly, for blood oxygenation measurements, the intensities of Fp fluorescence at two wavelengths are normalised with respect to the Fp fluorescence intensity at an isosbestic point, typically at a wavelength of about 530 nm.

Regarding fluorescence excitation for Fp, if only blood flow rate and/or only Fp concentration are to be monitored, and there is no need for the blood oxygenation parameter and/or for the blood volume parameter to be monitored, then any Fp excitation wavelength can be used, and does not need to be restricted to an isosbestic wavelength. Indeed as far as the Fp measurements are concerned, the reflection is measured, and used for correcting for the haemodynamic artifact, but the reflection measurements will not correctly represent blood volume changes since they will be influenced by blood oxygenation. However, it is important to provide a reflection that represents the blood volume, and for this reflectance must be measured when excitation is at an isosbestic wavelength. Thus the Fp excitation radiation is preferably chosen to be at an isosbestic wavelength, typically about 455 nm.

Thus, other than the specific choice of laser illumination wavelength, and the choice of wavelengths for determining the Fp level, reflectance and blood oxygenation state, determination of the blood flow rate and of the set of tissue viability parameters—Fp, blood volume and blood oxygenation state—in the second embodiment is as described for the first embodiment, mutatis mutandis, and thus the LSU (1), DTU(3), LSC (7), and to a lesser extent the EU (4), PC (5) and PS (6) need to be correspondingly adapted accordingly to take into account the different illuminating wavelength and the different range of fluorescent emission and reflection wavelengths obtained.

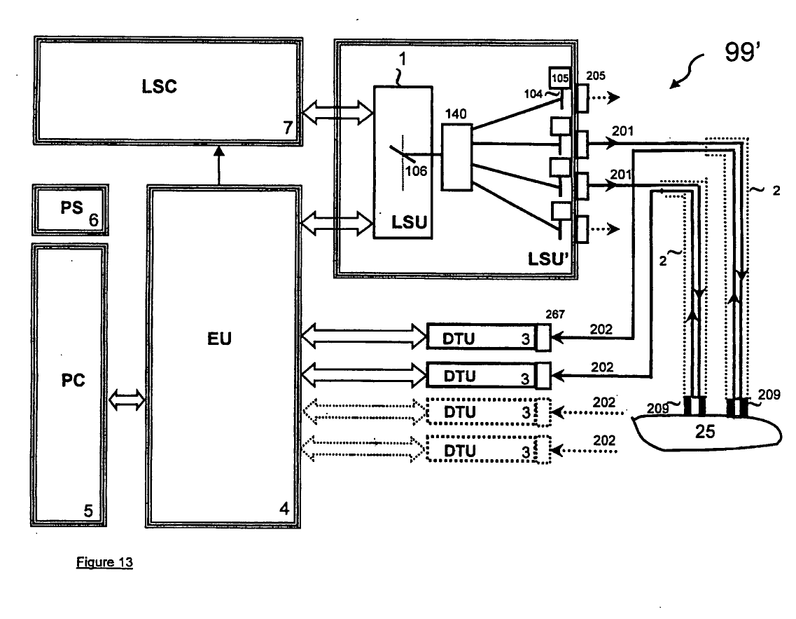

In some clinical procedures it is desirable to monitor the blood parameters for the assessment of organ tissue vitality in different regions of the body. In these situations, a multiple probe system is desirable. By way of example, a third embodiment of the present invention, is directed to a multi-probe system (99′), illustrated in FIG. 13. The system (99′) comprises at least two and preferably a plurality probes (2), each probe (2) being substantially the same as those described above with respect to the first embodiment. It will be appreciated however, that a plurality of probes (2) according to the second embodiment could be used, or, alternatively one or more probes according to each one of the first and second embodiments, mutatis mutandis may be used. Clearly, any given probe (2) comprised in the system (99′) may be adapted to monitor the same or different parameters to those monitored by any one of the other probes (2) thereof.

The chopping feature, which provide advantages in minimising exposure of the probed tissue to dangerous illumination levels, also facilitates a diversion of the irradiation light to any one of a plurality of probes (2), and subsequent detection of the return signals therefrom, by a corresponding plurality of detection units (DTU) (3). Each DTU (3) may be sampled in a predefined sequence that is correlated with the appearance of excitation light at appropriate probe (2). In other words, the multiprobe system (99′) essentially multiplexes the light source towards each one of the plurality of probes (99′), located on different parts of the tissue or organs.

The system (99′) according to the third embodiment of the present invention thus comprises similar components as previous preferred embodiments, viz LSU (1) probes (2), DTU (3), EU (4) PC (5), PS (6), LSC (7) as described with respect to the first and second embodiments, mutatis mutandis, with the following exceptions. The LSU (1) of the third embodiment, as illustrated in FIG. 13, though substantially similar to the LSU of the first or second embodiments (FIG. 4), further comprises the additional feature that the excitation light is passed through an acousto-optic deflector (AOD) (140) before being coupled and deflected to a plurality of excitation fibers by a corresponding plurality of lenses (104), each one being mounted on one of a plurality of adapters (105). The LSU (1) of the third embodiment is thus connected by said plurality of excitation fibers to a corresponding plurality of fiber optic probes (2), each probe (2) being coupled via an optical connector (205). In the third embodiment, the collecting fiber (202) from each probe (2) is thus connected to its corresponding DTU (3) by a corresponding optical connector (267) that is essentially similar to that used in the first and second embodiments.

From the optical coupler (267) the light passes to all the necessary components of the DTU (3), in a similar manner to that described for the first or second embodiment mutatis mutandis. Appropriate modification to the conditioning electronics of the EU (4) and the software running on the PC (5) is described below.

It should also be noted that multi-tissue element monitoring could also be accomplished by a plurality of probes (2), each one having a dedicated light source (LSU) with each probe unit being controlled by the same PC and EU units, and being powered using the same PS.

The EU (4) of the third embodiment is typically very similar to that of first or second embodiments. However, it further enables controlling of plurality of DTU (3) while the AOD (140) provides the excitation illumination each time to the appropriate probe (2). The electronics circuitry of the EU (4) is essentially the same as for the first and second embodiments.

The software running on the PC (5) is also typically very similar in concept to that described for first and second embodiments. However, the software also enables the two operation modes of third embodiment, as described hereinafter.

The third embodiment may be operated in a variety of modes as required by the clinical situation and diagnostic needs to which it is applied. Two particular modes of monitoring for which such multiple probe systems can be usefully applied, are described:

In the first mode, the mean signal intensities from the multiplicity of probes is calculated and displayed. This results in the parameters detected representing an average response of the multiplicity of tissue volumes probed, and will generally, better reflect the state of the organ layer (comprising the tissue volumes) as a whole. This mode of monitoring could be useful in transplantation surgery when better monitoring of the viability of donated organs are needed.

In the second mode, by applying one or several of the plurality of probes to each of several locations on the same organ or several different locations of different organs, the quasi-continuous monitoring of these organs over the same time period can be achieved by multiplexing the signals from the individual probes, with the parametric response of each organ being separately monitored and displayed.

The electronics and the software for the first mode will be substantially similar to that described with respect to the first or second embodiment. The main difference being that the chopping sequences used, and the sampling rate per probe, are engineered and optimized depending on number of probes, patient condition, and tissue type under observation.

The tₒₙ period per probe (2) of the system (99′) remains substantially the same as for the single probe of the first and second embodiments. However, during the OFF period for any probe in the system (99′), other probes (2) of the system may be selectively excited and measured. Accordingly, while the timing of the AOD (140) for each of the probes (2) in the system (99′) may be correlated with the sequence shown in FIG. 11(a), in practice the OFF period corresponding to any one probe is intercalated with the ON periods of the other probes, so that each subsequent pulse is delivered to the subsequent probe. After appropriate smoothing, the output signals from each DTU are used to generate a value for the desired blood viability parameter, corresponding of the mean value for the plurality of monitored tissue volumes, and thus more representative of the viability of the organ as a whole.

The second measurement mode of the third embodiment requires the same chopping sequence as that required by the first mode. In that second measurements mode the data acquired by each one of the DTUs will be treated and displayed by the PC separately.

Since the whole information, that is all signals for each probe is available in the computer, the signal from each probes can be processed separately, allowing the vitality parameters of each monitored tissue volume, corresponding to different organs to be monitored and displayed on the screen.

While specific embodiments of the invention have been described for the purpose of illustration, it will be understood that the invention may be carried out in practice by skilled persons with many modifications, variations and adaptations, without departing from its spirit or exceeding the scope of the claims.

1. (original) apparatus for selectively monitoring a blood flow rate tissue viability parameter and at least one second tissue viability parameter corresponding to a substantially identical tissue element, the apparatus comprising:—
illumination means for illuminating at least said tissue element with an illuminating radiation via at least one illumination location with respect to said tissue element;
radiation receiving means for receiving a radiation from said tissue element as a result of an interaction between said illuminating radiation and said tissue element, wherein a part of said received radiation is correlated to said blood flow rate tissue viability parameter, and wherein another part of said received radiation is correlated to said at least one second tissue viability parameter, said radiation receiving means being displaced from said illumination location by a displacement;
characterised in that said illuminating radiation is a laser radiation having a nominal wavelength in the range from about 370 nm to about 470 nm.

2. Apparatus as claimed in claim 1, wherein said laser radiation is generated in stable single longitudinal mode, wherein said nominal wavelength comprises a single waveband element.

3. Apparatus as claimed in claim 1, wherein said laser radiation is generated in two stable longitudinal non-competing modes, wherein said nominal wavelength comprises two discrete waveband elements.

4. Apparatus as claimed in claim 2, wherein said waveband element comprises a bandwidth of about 20 MHz, preferably about 10 MHz, more preferably about 6 MHz, and more preferably about 4 MHz.

5. Apparatus as claimed in claim 3, wherein said waveband elements each comprise a bandwidth of about 20 MHz, preferably about 10 MHz, more preferably about 6 MHz, and more preferably about 4 MHz.

6. Apparatus as claimed in claim 1, wherein said illumination location is provided by at least one excitation optical fiber having a free end capable of being brought into registry with said tissue element.

7. Apparatus as claimed in claim 6, wherein said radiation receiving means comprises at least one suitable receiving optical fiber having a free end capable of being brought into registry with said tissue element.

8. Apparatus as claimed in claim 7, wherein said at least one excitation optical fiber and said at least one receiving optical fiber are housed in a suitable probe head, wherein said free end of said at least one excitation fiber and said free end of said at least one receiving fiber are comprised on a contact face of said probe.

9. Apparatus as claimed in claim 8, wherein said at least one excitation fiber comprises a suitable first connector at an end thereof opposed to said free end thereof, said first connector capable of selectively coupling and decoupling said excitation fiber from the rest of the said apparatus.

10. Apparatus as claimed in claim 9, wherein said at least one collection fiber comprises a suitable second connector at an end thereof opposed to said free end thereof, said second connector capable of selectively coupling and decoupling said collection fiber from the rest of the said apparatus.

11. Apparatus as claimed in claim 8, wherein said probe is disposable.

12. Apparatus as claimed in claim 10, wherein said probe is sterilisable.

13. Apparatus as claimed in claim 1, wherein said illumination means comprises a suitable external cavity laser diode system.

14. Apparatus as claimed in claim 13, wherein said external cavity laser diode system is based on a suitable violet laser diode having an operating wavelength in the range of between about 370 nm and about 470 nm.

15. Apparatus as claimed in claim 14, wherein said external cavity laser diode system is configured according to the Littrow design.

16. Apparatus as claimed in claim 14, wherein said external cavity laser diode system is configured according to the Metcalf-Littman design.

17. Apparatus according to claim 14, wherein said external cavity diode laser system comprises a laser stabilisation control system for substantially preventing operation of the said external cavity diode laser system at mode competition conditions.

18. Apparatus according to claim 17, wherein said laser stabilisation control system is adapted for monitoring the laser intensity of the said external cavity laser diode system at a predetermined input current to said external cavity laser diode system and providing an electrical signal representative of said intensity, for varying the said input current within a predetermined range to provide corresponding electrical signals correlated to the resulting laser intensities generated, for identifying the corresponding electrical signal providing minimum RIN noise levels, and for adjusting the said input current such as to provide and maintain said electrical signal providing minimum RIN noise levels.

19. Apparatus as claimed in claim 1, wherein said blood flow rate tissue viability parameter is provided by applying a laser Doppler flowmetry technique to said radiation received by said radiation receiving means.

20. Apparatus as claimed in claim 19, further comprising first detection means for detecting said received radiation received by said radiation receiving means.

21. Apparatus as claimed in claim 1, wherein said illumination means is adapted to provide said illuminating radiation in pulses of predetermined duration and intensity by correspondingly chopping the illuminating radiation generated by said illuminating means.

22. Apparatus as claimed in claim 21, further comprising suitable control means for controlling the frequency of pulsing of said pulses.

23. Apparatus as claimed in claim 22, wherein said control means is further adapted to provide said pulses in packages of pulses, each package comprising at least one pulse and separated from a preceding or following package by a predetermined time period.

24. Apparatus as claimed in claim 23, wherein said predetermined time period is greater than the time interval between consecutive pulses within a package.

25. Apparatus as claimed in claim 23, wherein said time period is controllably variable.

26. Apparatus as claimed in claim 23, wherein the number of pulses within each package is controllably variable.

27. Apparatus as claimed in claim 1, wherein said nominal wavelength is at wavelength within the NADH excitation spectrum.

28. Apparatus as claimed in claim 27, wherein said nominal wavelength is at a suitable oxy-deoxy isobestic wavelength within the NADH excitation spectrum.

29. Apparatus as claimed in claim 28, wherein said nominal wavelength is about 390 nm±5 nm.

30. Apparatus as claimed in claim 28, wherein a said at least one second tissue viability parameter is NADH concentration, wherein said radiation received by said radiation receiving means comprises an NADH fluorescence emitted by the tissue in response to illumination thereof by said illuminating radiation, said at least one second tissue viability parameter being provided by the intensity of said NADH fluorescence.

31. Apparatus as claimed in claim 30, further comprising second detection means for detecting said received radiation received by said radiation receiving means.

32. Apparatus as claimed in claim 31, wherein said control means is operatively connected to said second detection means.

33. Apparatus as claimed in claim 32, wherein said control means is selectively responsive to previously detected signals corresponding to the detection of said received radiation detected by means of said second detection means of a prior monitoring cycle.

34. Apparatus as claimed in claim 28, wherein a said at least one second tissue viability parameter is blood volume within said tissue element, and said corresponding radiation received by said radiation receiving means comprises a reflection from the tissue element in response to illumination thereof by said illuminating radiation, the said at least one second tissue viability parameter being provided by the intensity of said reflection.

35. Apparatus as claimed in claim 34, further comprising third detection means for detecting said received radiation received by said radiation receiving means.

36. Apparatus as claimed in claim 35, wherein said control means is operatively connected to said third detection means.

37. Apparatus as claimed in claim 36, wherein said control means is selectively responsive to previously detected signals corresponding to the detection of said received radiation detected by means of said third detection means of a prior monitoring cycle.

38. Apparatus as claimed in claim 28, wherein a said at least one second tissue viability parameter is blood oxygenation ratio within said tissue element, and said corresponding radiation received by said radiation receiving means is a fluorescence emitted by the tissue in response to illumination thereof by said illuminating radiation, said at least one second tissue viability parameter being provided by the intensity of said fluorescence at least at two fluorescent emission wavelengths.

39. Apparatus as claimed in claim 38, wherein one of said at least two fluorescent wavelengths is chosen to lie at an oxy-deoxy isosbestic point of the NADH fluorescence emission spectrum.

40. Apparatus as claimed in claim 38, wherein one of said at least two fluorescent wavelengths is higher and another one of said at least two fluorescent wavelengths is smaller than a wavelength corresponding to an oxy-deoxy isosbestic point of the NADH fluorescence emission spectrum.

41. Apparatus as claimed in claim 40, wherein said blood oxygenation ratio parameter is provided by normalising said fluorescent intensities at said two wavelengths with respect to the fluorescent emission intensity at said oxy-deoxy isosbestic point of said NADH fluorescence emission spectrum.

42. Apparatus as claimed in claim 41, wherein said wavelength corresponding to said isosbestic point is about 455 nm±5 nm.

43. Apparatus as claimed in claim 40, further comprising fourth detection means for detecting said received radiation received by said radiation receiving means.

44. Apparatus as claimed in claim 40, wherein said control means is operatively connected to said fourth detection means.

45. Apparatus as claimed in claim 44, wherein said control means is selectively responsive to previously detected signals corresponding to the detection of said received radiation detected by means of said fourth detection means of a prior monitoring cycle.

46. Apparatus as claimed in claim 1, wherein said nominal wavelength is at wavelength within the Fp excitation spectrum.

47. Apparatus as claimed in claim 46, wherein said nominal wavelength is at a suitable oxy-deoxy isobestic wavelength within the Fp excitation spectrum.

48. Apparatus as claimed in claim 47, wherein said nominal wavelength is about 455 nm±5 nm.

49. Apparatus as claimed in claim 47, wherein a said at least one second tissue viability parameter is Fp concentration, wherein said radiation received by said radiation receiving means comprises an Fp fluorescence emitted by the tissue in response to illumination thereof by said illuminating radiation, said at least one second tissue viability parameter being provided by the intensity of said Fp fluorescence.

50. Apparatus as claimed in claim 49, further comprising second detection means for detecting said received radiation received by said radiation receiving means.

51. Apparatus as claimed in claim 50, wherein said control means is operatively connected to said second detection means.

52. Apparatus as claimed in claim 51, wherein said control means is selectively responsive to previously detected signals corresponding to the detection of said received radiation detected by means of said second detection means of a prior monitoring cycle.

53. Apparatus as claimed in claim 47, wherein a said at least one second tissue viability parameter is blood volume within said tissue element, and said corresponding radiation received by said radiation receiving means comprises a reflection from the tissue element in response to illumination thereof by said illuminating radiation, the said at least one second tissue viability parameter being provided by the intensity of said reflection.

54. Apparatus as claimed in claim 53, further comprising third detection means for detecting said received radiation received by said radiation receiving means.

55. Apparatus as claimed in claim 54, wherein said control means is operatively connected to said third detection means.

56. Apparatus as claimed in claim 55, wherein said control means is selectively responsive to previously detected signals corresponding to the detection of said received radiation detected by means of said third detection means of a prior monitoring cycle.

57. Apparatus as claimed in claim 47, wherein a said at least one second tissue viability parameter is blood oxygenation ratio within said tissue element, and said corresponding radiation received by said radiation receiving means is a fluorescence emitted by the tissue in response to illumination thereof by said illuminating radiation, said at least one second tissue viability parameter being provided by the intensity of said fluorescence at least at two fluorescent emission wavelengths.

58. Apparatus as claimed in claim 57, wherein one of said at least two fluorescent wavelengths is chosen to lie at an oxy-deoxy isosbestic point of the Fp fluorescence emission spectrum.

59. Apparatus as claimed in claim 57, wherein one of said at least two fluorescent wavelengths is higher and another one of said at least two fluorescent wavelengths is smaller than a wavelength corresponding to an oxy-deoxy isosbestic point of the Fp fluorescence emission spectrum.

60. Apparatus as claimed in claim 59, wherein said blood oxygenation ratio parameter is provided by normalising said fluorescent intensities at said two wavelengths with respect to the fluorescent emission intensity at said oxy-deoxy isosbestic point of said Fp fluorescence emission spectrum.

61. Apparatus as claimed in claim 60, wherein said wavelength corresponding to said isosbestic point is about 530 nm±5 nm.

62. Apparatus as claimed in claim 59, further comprising fourth detection means for detecting said received radiation received by said radiation receiving means.

63. Apparatus as claimed in claim 59, wherein said control means is operatively connected to said fourth detection means.

64. Apparatus as claimed in claim 63, wherein said control means is selectively responsive to previously detected signals corresponding to the detection of said received radiation detected by means of said fourth detection means of a prior monitoring cycle.

65. Apparatus as claimed in claim 46, wherein said nominal wavelength is about 440 mm±5 nm.

66. Apparatus as claimed in claim 22, wherein said control means is operatively connected to said first detection means.

67. A system for selectively monitoring at least two tissue viability parameter at a plurality of tissue elements; said system comprising a plurality of monitoring probes, each said probe comprising an apparatus as claimed in claim 1.

68. A system as claimed in claim 67, wherein at least two said probes are adapted for monitoring said tissue viability parameters of tissue elements within the same organ.

69. A system as claimed in claim 67, wherein at least two said probes are adapted for monitoring said tissue viability parameters of tissue elements within different organs.

70. A system as claimed in claim 69, wherein different organs are different organs within the same organism.

71. A system as claimed in claim 69, wherein different organs are different organs within different organisms.

72. A system as claimed in claim 69, wherein different organs are different organs include donor organs.

73. A system as claimed in claim 67, wherein said illuminating radiation for each said probe is provided by a common suitable light source.

74. A system as claimed in claim 73, wherein said first light source is a laser light source.

75. A system as claimed in claim 74, wherein said laser light source is adapted to provide said first illuminating radiation of said first wavelength in second pulses of predetermined duration and intensity.

76. A system as claimed in claim 75, further comprising suitable control means for controlling the frequency of pulsing of said second pulses.

77. A system as claimed in claim 76, wherein said control means is further adapted to provide said second pulses in packages of pulses, each package comprising at least one second pulse and separated from a preceding or following package by a predetermined time period.

78. A system as claimed in claim 77, wherein said predetermined time period is greater than the time interval between consecutive pulses within a package.

79. A system as claimed in claim 78, wherein said time period is controllably variable.

80. A system as claimed in claim 77, wherein the number of second pulses within each package is controllably variable.

81. A system as claimed in claim 77, wherein said control means is adapted for selectively directing discrete said second pulses to any one of said probes.
